(19) United States
(12) Patent Application Publication
(10) Pub. No.: US 2022/0370499 A1
(43) Pub. Date: Nov. 24, 2022

(54) IMMUNOTHERAPIES FOR TARGETING OF TUMOR VASCULATURE

(71) Applicant: Therapeutic Solutions International, Inc., Oceanside, CA (US)

(72) Inventors: Thomas E. Ichim, Oceanside, CA (US); Famela Ramos, Oceanside, CA (US); James Veltmeyer, Oceanside, CA (US); Timothy G. Dixon, Oceanside, CA (US); Feng Lin, Oceanside, CA (US); Wais Kaihani, Oceanside, CA (US)

(73) Assignee: Therapeutic Solutions International, Inc., Oceanside, CA (US)

(21) Appl. No.: 17/748,997

(22) Filed: May 19, 2022

Related U.S. Application Data
(60) Provisional application No. 63/192,207, filed on May 24, 2021.

(51) Int. Cl. A61K 35/17; C12N 5/0784; C12N 5/0789; A61K 39/00; A61P 37/06

(52) U.S. Cl. CPC A61K 35/17; C12N 5/0639; C12N 5/0647; A61K 39/0011; A61P 37/06; A61K 2039/5154

(57) ABSTRACT

Disclosed are novel means, protocols, and compositions of matter for creating targeted immune responses and/or induction of immunological memory towards the tumor vasculature. In one embodiment pluripotent stem cells are transfected with one or more genes capable of eliciting immunity, induced to differentiate into endothelial-like cells which resemble the tumor endothelial cells, and utilized as a vaccine. In some embodiment's genes are engineered under control of specific promoters to allow for various specificities of activity. In one specific embodiment pluripotent stem cells engineered to endow properties capable of inducing expression of the α-Gal epitope (Galα1,3Galα1,4GlcNAc-R). Addition of adjuvants to enhance antigen presentation of the vaccine composition, as well as means of stimulating systemic enhancement of circulating endothelial specific T cells are also disclosed.

### CROSS-REFERENCE TO RELATED APPLICATIONS

This application claims the benefit of priority to U.S. Provisional Application No. 63/192,207, filed May 24, 2021, the entire contents of which is incorporated herein by reference.

### FIELD OF THE INVENTION

The invention pertains to the treatment, prevention, or amelioration of tumor vasculature by using immune modulation of regenerative cells.

### BACKGROUND OF THE INVENTION

The angiogenic process is a very dynamic and fluid process. Although VEGF is critical in formation of new blood vessels, there are multiple other cytokines that possess redundant roles. Many times resistance to VEGF signaling is a result of tumor endothelial cells becoming more reliant on other cytokines that take over the function of VEGF pathway [1, 2]. Additionally, mutations in VEGF-R2 have been identified in tumor endothelium, which has been associated with non-responsiveness [3]. Dose escalation of VEGF inhibiting antibodies is limited by different toxicities such as cardiac toxicities [4-10]. An interesting observation is that although hematopoietic stem cells are known to utilize VEGF-R2 for self-renewal, cytopenias are generally not observed in patients receiving VEGF pathway blockers [11, 12]. Kinase inhibitors also suffer from mutations of active sites, as well as off target toxicities. For example, a study in colon patients receiving sunitinib demonstrated mutations in all major kinases associated with endothelial proliferation [13].

Thus limitations of efficacy of anti-angiogenesis approaches that are currently in the clinic appear to be associated with: a) targeting of only one pathway allows the tumor endothelium to start utilizing other pathways; and b) small molecule inhibitors are slow in onset of action, which allows for time to pass and mutations to accumulate.

Immunological targeting of angiogenesis may be a more promising approach due to: a) ability of immune system to “mutate” with cancer endothelium, thus overcoming ability of molecular evasion; b) more rapid onset of immune attack, including direct killing of endothelium may not allow enough time for tumor endothelium to mutate and/or acquire resistant properties.

### SUMMARY

Preferred embodiments include methods of inducing a T cell and/or B cell response to tumor associated endothelial cells and/or tumor vascular channels comprising the steps of: a) selecting a pluripotent stem cell; b) modifying said pluripotent stem cell so as to induce expression of an immunogenic molecule; c) modifying said pluripotent stem cell so as to induce loss of expression of an immunosuppressive molecule; d) modifying said pluripotent stem cell so as to induce loss of expression of an immune suppressive signaling molecule; e) inducing differentiation of said pluripotent stem cell into endothelial cells under conditions which replicate the tumor microenvironment; f) obtaining said endothelial cells differentiated under conditions that replicate said tumor microenvironment and substantially isolating said cells in order to obtain a relatively homogeneous population of cells which resemble tumor endothelium associated cells; g) optionally mitotically inactivating said cells; and h) administering said cells in a manner to stimulate an immune response.

Preferred methods include embodiments wherein said endothelial-like cell as transfected with GM-CSF.

Preferred methods include embodiments wherein said transfection with GM-CSF is sufficient to induce differentiation of dendritic cells in proximity to said administered endothelial-like cell.

Preferred methods include embodiments wherein said dendritic cells express CD40.

Preferred methods include embodiments wherein said dendritic cells express CD11c.

Preferred methods include embodiments wherein said dendritic cells express CD83.

Preferred methods include embodiments wherein said dendritic cells express CD80.

Preferred methods include embodiments wherein said dendritic cells express CD86.

Preferred methods include embodiments wherein said endothelial-like cells are co-administered with monocytes.

Preferred methods include embodiments wherein said monocytes are allogeneic to the recipient.

Preferred methods include embodiments wherein said monocytes are autologous to the recipient.

Preferred methods include embodiments wherein said monocytes are xenogeneic to the recipient.

Preferred methods include embodiments wherein said monocytes are transfected with interleukin-1.

Preferred methods include embodiments wherein said monocytes are induced to produce interleukin-1.

Preferred methods include embodiments wherein said monocytes are induced to produce interleukin-1 by incubation with a toll like receptor agonist.

Preferred methods include embodiments wherein said toll like receptor is toll like receptor 4.

Preferred methods include embodiments wherein said toll like receptor 4 is activated by lipopolysaccharide.

Preferred methods include embodiments wherein said toll like receptor 4 is activated by HMGB -1.

Preferred methods include embodiments wherein said toll like receptor 4 is activated by beta glucan.

Preferred methods include embodiments wherein said toll like receptor 4 is activated by BCG.

Preferred methods include embodiments wherein said toll like receptor 4 is activated by neutrophil extracellular traps.

Preferred methods include embodiments wherein said immune response is an antibody mediated immune response.

Preferred methods include embodiments wherein said immune response is a cell mediated immune response.

Preferred methods include embodiments wherein said immune response is a natural killer cell mediated immune response.

Preferred methods include embodiments wherein said immune response is an NKT cell mediated immune response.

Preferred methods include embodiments wherein said immune response is a macrophage mediated immune response.

Preferred methods include embodiments wherein said tumor associated blood vessels are angiogenic.

Preferred methods include embodiments wherein said tumor associated blood vessels are vasculogenic.

Preferred methods include embodiments wherein said tumor associated blood vessels are comprised of proliferating cells.

Preferred methods include embodiments wherein said tumor associated blood vessels are comprised of proliferating endothelial cells.

Preferred methods include embodiments wherein said tumor associated blood vessels possess expression of TEM-1.

Preferred methods include embodiments wherein said tumor associated blood vessels possess expression of VEGF-receptor.

Preferred methods include embodiments wherein said tumor associated blood vessels possess expression of nestin.

Preferred methods include embodiments wherein said tumor associated blood vessels possess expression of TREM-1.

Preferred methods include embodiments wherein said tumor associated blood vessels possess expression of CD31.

Preferred methods include embodiments wherein said tumor associated blood vessels possess expression of vWF.

Preferred methods include embodiments wherein said tumor associated blood vessels possess expression of CD133.

Preferred methods include embodiments wherein said tumor associated blood vessels possess expression of CD34.

Preferred methods include embodiments wherein said tumor associated blood vessels possess expression of CD133.

Preferred methods include embodiments wherein said tumor associated blood vessels possess expression of Factor VIII.

Preferred methods include embodiments wherein said tumor associated blood vessels possess expression of c-met.

Preferred methods include embodiments wherein said tumor associated blood vessels possess expression of PDGF-BB receptor.

Preferred methods include embodiments wherein said tumor associated blood vessels possess expression of EGF-receptor.

Preferred methods include embodiments wherein said tumor associated blood vessels possess expression of FGF-1 receptor.

Preferred methods include embodiments wherein said tumor associated blood vessels possess expression of FGF-2 receptor.

Preferred methods include embodiments wherein said tumor associated blood vessels possess expression of klotho receptor.

Preferred methods include embodiments wherein said tumor associated blood vessels possess expression of netrin.

Preferred methods include embodiments wherein said tumor associated blood vessels possess expression of thrombopoietin receptor.

Preferred methods include embodiments wherein said tumor associated blood vessels possess expression of interleukin-3 receptor.

Preferred methods include embodiments wherein said tumor associated blood vessels possess expression of nestin.

Preferred methods include embodiments wherein said pluripotent stem cell is capable of forming a teratoma when placed in an immunodeficient mouse.

Preferred methods include embodiments wherein said pluripotent stem cell is capable of forming ectoderm, mesoderm and endoderm tissue.

Preferred methods include embodiments wherein said pluripotent stem cell is generated as an inducible pluripotent stem cell.

Preferred methods include embodiments wherein said pluripotent stem cell expresses OCT4.

Preferred methods include embodiments wherein said pluripotent stem cell expresses Sox-2.

Preferred methods include embodiments wherein said pluripotent stem cell expresses NANOG.

Preferred methods include embodiments wherein said pluripotent stem cell expresses SSEA4.

Preferred methods include embodiments wherein said pluripotent stem cell expresses c-myc.

Preferred methods include embodiments wherein said pluripotent stem cell expresses DNMT3B.

Preferred methods include embodiments wherein said pluripotent stem cell expresses KLF4.

Preferred methods include embodiments wherein said pluripotent stem cell expresses Lin28.

Preferred methods include embodiments wherein said pluripotent stem cell expresses PRDM14.

Preferred methods include embodiments wherein said pluripotent stem cell expresses SALL4.

Preferred methods include embodiments wherein said pluripotent stem cell expresses SSEA1.

Preferred methods include embodiments wherein said pluripotent stem cell expresses SSEA3.

Preferred methods include embodiments wherein said pluripotent stem cell expresses TRA-1-60.

Preferred methods include embodiments wherein said pluripotent stem cell expresses TRA-1-81.

Preferred methods include embodiments wherein said pluripotent stem cell is derived by somatic cell nuclear transfer.

Preferred methods include embodiments wherein said pluripotent stem cell is derived by parthenogenesis.

Preferred methods include embodiments wherein said pluripotent stem cell is derived by exposure to acid.

Preferred methods include embodiments wherein said pluripotent stem cell is transfected with an immune stimulatory cytokine whose expression is either constitutive or inducible.

Preferred methods include embodiments wherein said immune stimulatory cytokine is RANTES.

Preferred methods include embodiments wherein said immune stimulatory cytokine is MIP-1 alpha.

Preferred methods include embodiments wherein said immune stimulatory cytokine is MIP-1 beta.

Preferred methods include embodiments wherein said immune stimulatory cytokine is interleukin-1.

Preferred methods include embodiments wherein said immune stimulatory cytokine is interleukin-2.

Preferred methods include embodiments wherein said immune stimulatory cytokine is interleukin-6.

Preferred methods include embodiments wherein said immune stimulatory cytokine is interleukin-7.

Preferred methods include embodiments wherein said immune stimulatory cytokine is interleukin-8.

Preferred methods include embodiments wherein said immune stimulatory cytokine is interleukin-9.

Preferred methods include embodiments wherein said immune stimulatory cytokine is interleukin-12.

Preferred methods include embodiments wherein said immune stimulatory cytokine is interleukin-15.

Preferred methods include embodiments wherein said immune stimulatory cytokine is a CCR5 agonist.

Preferred methods include embodiments wherein said immune stimulatory cytokine is interleukin-17.

Preferred methods include embodiments wherein said immune stimulatory cytokine is interleukin-18.

Preferred methods include embodiments wherein said immune stimulatory cytokine is interleukin-20.

Preferred methods include embodiments wherein said immune stimulatory cytokine is interleukin-22.

Preferred methods include embodiments wherein said immune stimulatory cytokine is TNF-alpha.

Preferred methods include embodiments wherein said immune stimulatory cytokine is TNF-beta.

Preferred methods include embodiments wherein said immune stimulatory cytokine is interferon alpha.

Preferred methods include embodiments wherein said immune stimulatory cytokine is interferon beta.

Preferred methods include embodiments wherein said immune stimulatory cytokine is interferon gamma.

Preferred methods include embodiments wherein said immune stimulatory cytokine is interferon epsilon.

Preferred methods include embodiments wherein said immune stimulatory cytokine is interferon tau.

Preferred methods include embodiments wherein said immune stimulatory cytokine is interferon omega.

Preferred methods include embodiments wherein said immune stimulatory cytokine is CD80.

Preferred methods include embodiments wherein said immune stimulatory cytokine is CD86.

Preferred methods include embodiments wherein said immune stimulatory cytokine is CD40.

Preferred methods include embodiments wherein said immune stimulatory cytokine is CD40 ligand.

Preferred methods include embodiments wherein said immune stimulatory cytokine is CD2.

Preferred methods include embodiments wherein said immune stimulatory cytokine is CD5.

Preferred methods include embodiments wherein said immune stimulatory cytokine is CD28.

Preferred methods include embodiments wherein said immune stimulatory cytokine is Fas ligand.

Preferred methods include embodiments wherein said immune stimulatory cytokine is G-CSF.

Preferred methods include embodiments wherein said immune stimulatory cytokine is GM-CSF.

Preferred methods include embodiments wherein said immune stimulatory cytokine is M-CSF.

Preferred methods include embodiments wherein said immune stimulatory cytokine is lymphotactin.

Preferred methods include embodiments wherein said immune stimulatory cytokine is SDF-1.

Preferred methods include embodiments wherein said pluripotent stem cell is transfected with alpha1,3-galactosyltransferase.

Preferred methods include embodiments wherein said pluripotent stem cell is gene edited to remove expression of IL-10 receptor.

Preferred methods include embodiments wherein said pluripotent stem cell is gene edited to remove expression of IL-4 receptor.

Preferred methods include embodiments wherein said pluripotent stem cell is gene edited to remove expression of IL-3 receptor.

Preferred methods include embodiments wherein said pluripotent stem cell is gene edited to remove expression of NGF receptor.

Preferred methods include embodiments wherein said pluripotent stem cell is gene edited to remove expression of TNF receptor p55.

Preferred methods include embodiments wherein said pluripotent stem cell is gene edited to remove expression of TNF receptor p75.

Preferred methods include embodiments wherein said endothelial cells are generated by culture for pluripotent stem cells in a media inductive of CD133 generation.

Preferred methods include embodiments wherein said pluripotent stem cells are cultured in PDGF-BB to generate endothelial progenitor cells.

Preferred methods include embodiments wherein said pluripotent stem cells are cultured in PDGF-BB to generate endothelial progenitor cells.

Preferred methods include embodiments wherein said immunogenicity is augmented by fusion of human pluripotent cell with a cell from an animal that is not a human or a primate.

Preferred methods include embodiments wherein said endothelial cells are generated from said pluripotent cell by: a) culturing or maintaining a plurality of substantially undifferentiated pluripotent cells in a defined media comprising at least one growth factor; b) culturing the pluripotent cells in a defined media comprising an amount of BMP4 and VEGF sufficient to expand or promote differentiation in a plurality of the cells; and c) culturing the cells of (b) in a defined media comprising an amount of either (1) IL-3 and Flt3 ligand, or (2) VEGF, FGF-2 or an FGF-2 mimic, and IGF sufficient to further expand or promote differentiation in a plurality of the cells; wherein a plurality of the pluripotent cells are differentiated into hematopoietic precursor cells or endothelial cells.

Preferred methods include embodiments wherein the defined media of step (b) further comprises FGF-2 or an FGF-2 mimic.

Preferred methods include embodiments wherein the defined media of step (c) comprises IL-3, Flt3 ligand, and GMCSF.

Preferred methods include embodiments wherein the defined media of step (c) comprises IL-3, Flt3 ligand, and at least one of IL-6, SCF, or TPO.

Preferred methods include embodiments wherein the defined media of step (c) further comprises IL-6, SCF, and TPO.

Preferred methods include embodiments wherein the defined media of stem (c) further comprises IL-2.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is a bar graph showing that the administration of StemVacs-V iPSC+Poly IC were highly effective in shrinking tumors.

### DETAILED DESCRIPTION OF THE INVENTION

The invention provides means of augmenting the induction immune responses towards tumor endothelial cells by providing adjuvants useful for enhancing immunization with pluripotent stem cell derived endothelial cells generated in a manner to replicate immunogenic characteristics of tumor endothelial cells. In some situations adjuvants for the practice of the invention are antiangiogeneic compounds. The concept of treating cancer by blocking new blood vessel formation, angiogenesis, was pioneered by Judah Folkman who provided convincing arguments that it is not necessary to actively kill the tumor mass, but by suppressing its ability to grow through cutting off blood supply, malignant tumors may be converted into benign masses that eventually regress [14, 15]. Unfortunately, despite discovery of angiostatin, and endostatin, naturally derived inhibitors of angiogenesis, neither of these approaches translated into successful therapies (http://www.nytimes.com/1998/11/13/us/a-failure-to-verify-a-cancer-advance-is-raising-concern.html). Nevertheless, the concept of targeting new blood vessel formation led to thousands of publications describing various antiangiogenic agents, of which several eventually proceeded through clinical trials and regulatory approval. Broadly anti-angiogenic agents approved by regulators can be classified into antibodies, such as Bevacizumab (Avastin) which binds VEGF [16], and Ramucirumab (Cyramza) [17], which binds VEGF-R2, as well as small molecules which bind multiple receptor kinases associated with angiogenesis such as Sunitinib [18-20], Cabozantinib [21-24], Pazopanib [25-27], and Regorafenib [28-30]. In one embodiment, the invention teaches the use of iPSC derived tumor endothelial-like cells together with standard and experimental antiangiogenic agents.

The current antiangiogenic approaches have augmented the standard of care for various tumor types and have achieved some level of progress. Unfortunately, the concept of blocking angiogenesis of cancer was not as simple as originally envisioned. One of the major hurdles in blocking angiogenesis was that even though de novo blood vessels are derived from nonmalignant cells, the malignant cells appear to possess ability to induce mutations in the new blood vessels. One example of the heterogeneity of tumor endothelial cells compared to endothelial cells from low and high metastatic tumors by Ohga et al [31]. The invention teaches the use of standardized endothelial progenitor cells originating from iPSC cells in order to possess a uniform population of cells for stimulation of immunity towards the tumor endothelium. In some studies, the investigators extracted two types of tumor endothelial cells (TEM) from high-metastatic (HM) and low-metastatic (LM) tumors and compared their characteristics. HM tumor-derived TECs (HM-TECs) showed higher proliferative activity and invasive activity than LM tumor-derived TECs (LM-TECs). Moreover, the mRNA expression levels of pro-angiogenic genes, such as vascular endothelial growth factor (VEGF) receptors 1 and 2, VEGF, and hypoxia-inducible factor-1α, were higher in HM-TECs than in LM-TECs. The tumor blood vessels themselves and the surrounding area in HM tumors were exposed to hypoxia. Furthermore, HM-TECs showed higher mRNA expression levels of the stemness-related gene stem cell antigen and the mesenchymal marker CD90 compared with LM-TECs. HM-TECs were spheroid, with a smoother surface and higher circularity in the stem cell spheroid assay. HM-TECs differentiated into osteogenic cells, expressing activated alkaline phosphatase in an osteogenic medium at a higher rate than either LM-TECs or normal ECs. Furthermore, HM-TECs contained more aneuploid cells than LM-TECs. The investigators concluded that the results indicate that TECs from HM tumors have a more pro-angiogenic phenotype than those from LM tumors. It appears that the aggressiveness of the tumor not only can alter endothelial cell function but also drug resistance ability. In another study, Akiyama et al. [32]compared murine TECs and normal ECs. It was found that TECs were more resistant to paclitaxel with the up-regulation of multidrug resistance (MDR) 1 mRNA, which encodes the P-glycoprotein, compared with normal ECs. Normal human microvascular ECs were cultured in tumor-conditioned medium (CM) and became more resistant to paclitaxel through MDR1 mRNA up-regulation and nuclear translocation of Y-box-binding protein 1, which is an MDR1 transcription factor. Vascular endothelial growth factor (VEGF) receptor 2 (VEGFR2) and Akt were activated in human microvascular ECs by tumor CM. The investigators observed that tumor CM contained a significantly high level of VEGF. A VEGFR kinase inhibitor, Ki8751, and a phosphatidylinositol 3-kinase-Akt inhibitor, LY294002, blocked tumor CM-induced MDR1 up-regulation. MDR1 up-regulation, via the VEGF-VEGFR pathway in the tumor microenvironment, is one of the mechanisms of drug resistance acquired by TECs. It was observed that VEGF secreted from tumors up-regulated MDR1 through the activation of VEGFR2 and Akt. This process is a novel mechanism of the acquisition of drug resistance by TECs in the tumor microenvironment. Yet another study demonstrated that tumors can induce a “dedifferentiation” of tumor endothelium. Specifically, compared with NECs, stem cell markers such as Sca-1, CD90, and multidrug resistance 1 are upregulated in TECs, suggesting that stem-like cells exist in tumor blood vessels. TECs and NECs were isolated from melanoma-xenografted nude mice and normal dermis, respectively. The stem cell marker aldehyde dehydrogenase (ALDH) mRNA expression and activity were higher in TECs than those in NECs. Next, ALDHhigh/low TECs were isolated by fluorescence-activated cell sorting to compare their characteristics. Compared with ALDHlow TECs, ALDHhigh TECs formed more tubes on Matrigel-coated plates and sustained the tubular networks longer. Furthermore, VEGFR2 expression was higher in ALDHhigh TECs than that in ALDHlow TECs. In addition, ALDH was expressed in the tumor blood vessels of in vivo mouse models of melanoma and oral carcinoma, but not in normal blood vessels. These findings indicate that ALDHhigh TECs exhibit an angiogenic phenotype. Stem-like TECs may have an essential role in tumor angiogenesis [33].

What is it that causes the tumor to evoke changes in the endothelium? As suggested above, there is some support for growth factor mediated alterations, additionally, horizontal gene transfer may also play a role [34-42]. Although the field of horizontal gene transfer has historically been controversial one of the strongest evidences supporting this concept is the phenomena of donor-derived relapse in leukemic patients. In these situations patients with leukemia who relapse after bone marrow transplant have the relapsing cells originate from donor cells that transformed into malignant cells [43, 44]. Another issue that affected efficacy of anti-angiogenesis therapies is that in some tumors, the tumor cells themselves transdifferentiate into endothelial-like cells, termed tumor vascular channels, which possess ability to mutate around either antibody or kinase inhibitor drugs [45-50].

The previously mentioned means by which tumor endothelial cells can protect themselves against anti-angiogenic agents has resulted in relatively low clinical efficacy of these drugs. To understand the general lack of efficacy in the initial registration trial (https://clinicaltrials.gov/show/NCT00976911), median progression free survival (PFS) of ovarian cancer patients who received bevacizumab plus chemotherapy was 6.8 months (95 percent CI: 5.6, 7.8) compared with 3.4 months (95 percent CI: 2.1, 3.8) for those who received chemotherapy alone. There was no statistically significant difference in overall survival (OS) for patients treated with bevacizumab plus chemotherapy compared with chemotherapy alone (median OS: 16.6 months versus 13.3 months; HR 0.89; 95 percent CI: 0.69, 1.14). Subset analysis led to identification that the group of patients that received paclitaxel with the antibody had the largest improvement, resulting in a 5.7-month improvement in median PFS (9.6 months versus 3.9 months; HR 0.47; 95 percent CI: 0.31, 0.72), an improvement in the objective response rate (53 percent versus 30 percent), and a 9.2-month improvement in median OS (22.4 months versus 13.2 months, HR 0.64; 95 percent CI: 0.41, 1.01) (https://www.cancer.gov/about-cancer/treatment/drugs/fda-bevacizumab). Multiple other trials where conducted for different indications using bevacizumab, unfortunately, progression free survival and overall survival was not increase more than a year in any of the studies [51-55], and neither in studies with small molecule kinase inhibitors [56-61].

This clinical translation, although highly beneficial in some patients, overall the effect was mediocre, highlights the disparity between animal studies, in which some studies complete regression was observed in established tumors [62, 63], whereas in clinical trials, relatively minimal effect compared to animal studies was observed [64]. One lesson from these studies is that the large heterogeneity of the patient and of the tumors, which calls for large patient populations in order to achieve an overall survival advantage. Innovations in pharmacogenomics and personalized medicine will help identify specific patients and tumors that are likely to respond. Unfortunately, at present, patients with metastatic disease have limited options and a statistically significant extension of survival does equate to large market demand, as seen by the overall sale of angiogenesis inhibitors for cancer being over 20 billion annually.

In one embodiment inducible pluripotent stem cells are modified to increase immunogenicity and differentiated under conditions of hypoxia, acidosis, tryptophan depletion and other conditions known to be associated with the tumor microenvironment [65, 66]. Said iPSC generated tumor endothelial cells (iPSC-TEC) are made immunogenic by inducing expression of the α-Gal epitope (Galα1,3Galα1,4GlcNAc-R).

A media is said to be “essentially free” of a growth factor if the growth factor is absent from the media or if the growth factor is present in the media in an amount which is insufficient to promote any substantial expansion and/or differentiation of cells in the media, or is present at a concentration below a detectable limit. It will be recognized that a media which is essentially free of a growth factor may nonetheless contain trace amounts of the growth factor in the media.

The terms “inhibiting,” “reducing,” or “prevention,” or any variation of these terms, when used in the claims and/or the specification includes any measurable decrease or complete inhibition to achieve a desired result.

The term “effective,” as that term is used in the specification and/or claims, means adequate to accomplish a desired, expected, or intended result.

The use of the word “a” or “an” when used in conjunction with the term “comprising” in the claims and/or the specification may mean “one,” but it is also consistent with the meaning of “one or more,” “at least one,” and “one or more than one.”

It is contemplated that any embodiment discussed in this specification can be implemented with respect to any method or composition of the invention, and vice versa. Furthermore, compositions of the invention can be used to achieve methods of the invention.

Throughout this application, the term “about” is used to indicate that a value includes the inherent variation of error for the device, the method being employed to determine the value, or the variation that exists among the study subjects.

The use of the term “or” in the claims is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

An aspect of the present invention relates to a method of differentiating pluripotent cells into hematopoietic precursor cells or endothelial cells comprising the sequential steps of: (a) culturing or maintaining a plurality of substantially undifferentiated pluripotent cells in a first defined media comprising at least one growth factor, (b) incubating the cells in a second defined media which is essentially free of BMP4, VEGF, IL-3, Flt3 ligand, and GMCSF, (c) culturing the cells in a third defined media comprising an amount of BMP4 and VEGF sufficient to expand or promote differentiation in a plurality of the cells, and (d) culturing the cells in a fourth defined media comprising an amount of either (1) IL-3 and Flt3 ligand, or (2) VEGF, FGF-2 or an FGF-2 mimic, and IGF sufficient to expand or promote differentiation in a plurality of the cells; wherein a plurality of the pluripotent cells are differentiated into hematopoietic precursor cells or endothelial cells. In certain embodiments, combination (1) above may be used to promote differentiation into hematopoietic precursor cells. Combination (2) above may be used to promote differentiation into an endothelial cell or an endothelial progenitor cell. The second defined media may be free or essentially free of FGF-2, IL6, SCF and/or TPO. The third defined media may also include FGF-2 (e.g., from about 5-50 ng/ml or from about 10-25 ng/ml) or an FGF-2 mimic. As shown in the below examples, inclusion of FGF-2 in the third media can increase the efficiency of differentiation of pluripotent cells into hematopoietic precursor cells. In certain embodiments, the fourth defined media further comprises GMCSF, or at least one of IL-6, SCF, or TPO. In certain embodiments, the fourth defined media includes an amount of either: (1) IL-3, Flt3 ligand, and GMCSF, or (2) IL-3, Flt3 ligand, SCF, IL-6, and TPO sufficient to promote differentiation of the cells. The third defined media and/or the fourth defined media may further comprise BIT9500 or Serum Replacement 3. The method may comprise culturing cells in a defined media which includes BIT9500 or Serum Replacement 3. At least some of the cells may be at least partially separated or are substantially individualized prior to step (b). The cells may be substantially individualized using an enzyme, such as a trypsin. The cells may be contacted with a ROCK inhibitor and a trypsin inhibitor (e.g., a soybean trypsin inhibitor) subsequent to said individualization. The ROCK inhibitor may be selected from the list consisting of HA-100, H-1152, and Y-27632. A plurality of the pluripotent cells may form embryoid bodies (EBs). From about 200 to about 1000 cells per aggregate may be used to generate at least one of said EBs. The method may comprise culturing the cells at an atmospheric pressure of less than 20% oxygen or at an atmospheric pressure of about 5% oxygen. As shown in the below examples, differentiating cells under hypoxic conditions, such as at about 5% atmospheric O.sub.2, can increase the differentiation of the cells, e.g., into hematopoietic and/or endothelial precursor cells.

In certain embodiments, said cells may be partially or substantially reaggregated at least once. The cells may be reaggregated after culture in the third defined media and prior to or during culture in the fourth defined media. The reaggregation may comprise exposing said cells to trypsin or TRYPLE. Said cells may be exposed to a ROCK inhibitor subsequent to the reaggregation, or said cells may be cultured in a media essentially free of a ROCK inhibitor subsequent to the reaggregation. The method may further comprise culturing the cells at an atmospheric pressure of less than about 20% oxygen, wherein from about 200 to about 1000 cells per aggregate are used to generate a plurality of embryoid bodies (EBs). The first defined media may comprise TeSR, mTeSR, or mTeSR1. Step (a) may comprise culturing the cells on a matrix-coated surface. The matrix may comprise laminin, vitronectin, gelatin, polylysine, thrombospondin or Matrigel™. The second defined media may comprise TeSR-GF or X-vivo15 media. The second defined media may further comprise about 0.1 ng/ml TGF-.beta. and about 20 ng/ml FGF-2. Step (b) may comprise incubating the cells for a period of from about 12 hours to about 3 days. Step (c) may comprise culturing or differentiating the cells for a period of from about 4 to about 8 days. Step (d) may comprise culturing the cells for a period of at least about 4, or from about 4 to about 8 days. A plurality of the pluripotent cells may be differentiated into multipotent heamatopoietic, or myeloid progenitor cells. In certain embodiments, the myeloid progenitor cells co-express CD31, CD43, and CD45. The myeloid progenitor cells may be common myeloid progenitors. The third defined media comprises about 10-50 ng/ml BMP4 and about 10-50 ng/ml VEGF. In certain embodiments, the third defined media further comprises 10-50 ng/ml FGF-2. The third defined media comprises about 25 ng/ml BMP4 and about 25 ng/ml VEGF. The fourth defined media may comprise about 5-25 ng/ml IL-3 and about 10-50 ng/ml Flt3 ligand. The fourth defined media may further comprise about 5-25 ng/ml GMCSF, or about 10-100 ng/ml or about 10-50 ng/ml TPO, about 10-100 ng/ml SCF, about 5-25 ng/ml IL-6, and about 5-25 ng/ml IL-3. The fourth defined media may comprise about 10 ng/ml IL-3, about 25 ng/ml Flt3 ligand, and about 10 ng/ml GMCSF. A plurality of the hematopoietic precursor cells may express at least two cell markers selected from the list comprising CD43, CD34, CD31 and CD45. A plurality of the hematopoietic precursor cells may express CD34, CD43, CD45 and CD31. In certain embodiments, the hematopoietic precursor cells are multipotent hematopoietic precursor cells that co-express CD34, CD43, CD45 and CD31. In certain embodiments, a fifth defined media may be used to further promote differentiation of the cells into a particular cell type; for example, various media may be used to promote differentiation of the hematopoietic precursor cells into a more differentiated cell type such as, for example, an erythroblast, a NK cell, or a T cell. The method may further comprise culturing a plurality of said cells in a fifth defined media comprising one or more growth factor selected from the list consisting of IL-3, IL-6, SCF, EPO, and TPO, in an amount sufficient to promote differentiation of a plurality of the cells into erythroblasts. A plurality of the cells are cultured in a fifth defined media comprising one or more growth factor selected from the list consisting of IL-7, SCF, and IL-2, in an amount sufficient to promote differentiation of the cells into NK cells. The method may further comprise culturing a plurality of said cells in a fifth defined media comprising Notch ligand and one or more growth factor selected from the list consisting of IL-7, SCF, and IL-2 in an amount sufficient to promote differentiation of the cells into T cells. The Notch ligand may be the Fc chimeric Notch DLL-1 ligand or Notch ligand produced by a stromal cell line which over-expresses the Notch ligand. In certain embodiments, a thymic peptide such thymosin alpha, thymopenin, or thymosin B4 may be used to further promote differentiation of the cells into T cells (e.g., as described in Peng et al., 2008). In certain embodiments, the plurality of said cells comprise hematopoietic precursor cells. The third defined media may comprise one or more growth factor selected from the list consisting of SCF, IL-6, G-CSF, EPO, TPO, FGF2, IL-7, IL-11, IL-9, IL-13, IL-2, or M-CSF in an amount sufficient to promote expansion or further differentiation of the cells. The fourth defined media may comprise one or more growth factor selected from the list consisting of SCF, IL-6, G-CSF, EPO, TPO, FGF2, BMP4, VEGF, IL-7, IL-11, IL-9, IL-13, IL-2, or M-CSF in an amount sufficient to promote expansion or further differentiation of the cells. In certain embodiments, the method may comprise incubating the cells in a fifth defined media which includes one or more growth factor selected from the list consisting of SCF, IL-6, G-CSF, EPO, TPO, FGF2, IL-7, IL-11, IL-9, IL-13, IL-2, or M-CSF in an amount sufficient to promote expansion or further differentiation of the cells. Said pluripotent cells are preferably mammalian pluripotent cells. In certain embodiments the pluripotent cells are human pluripotent cells, such as human embryonic stem cells (hESC) or induced pluripotent cells (iPSC). The hESC comprise cells may be selected from the list consisting of H1, H9, hES2, hES3, hES4, hES5, hES6, BG01, BG02, BG03, HSF1, HSF6, H1, H7, H9, H13B, and H14. Said iPSC may be selected from the list consisting of iPS6.1, iPS 6.6, iPS, iPS 5.6, iPS iPS 5.12, iPS 5.2.15, iPS iPS 5.2.24, iPS 5.2.20, iPS 6.2.1, iPS-B1-SONL, iPS-B1-SOCK, iPS-TIPS 1EE, iPS-TiPS IB, iPS-KIPS-5, and iPS 5/3-4.3. Another aspect of the present invention related to hematopoietic precursor cell differentiated according to the methods described herein or derived from a separate hematopoietic precursor cell differentiated according to the methods described herein. The hematopoietic precursor cell may express two, three or all of CD34, CD43, CD45, and CD31. Yet another aspect of the present invention relates to a myeloid cell, a myeloid progenitor, or a common myeloid progenitor derived from a hematopoietic precursor cell differentiated according to the methods described herein. The myeloid cell may be selected from the list consisting of monocyte, macrophage, neutrophil, basophil, eosinophil, erythrocyte, megakaryocyte/platelet, and dendritic cell. In certain embodiments, the myeloid cell is an erythrocyte. The myeloid cell, myeloid progenitor, or the common myeloid progenitor may be comprised in a pharmaceutical preparation.

In one embodiment of the invention, administration of pluripotent stem cell derived tumor endothelial cells (iPSC-TEC) is performed in the presence of other immune activators. It is known from studies of immune modulators that recruitment of multiple arms of the immune system associates with increased efficacy. Accordingly, in one embodiment of the invention, numerous anti-cancer immune effector cells are stimulated together with administration of the iPSC-TEC immunotherapy. It is known that natural killer cells play an important role in immune destruction of cancer [67-73]. In one embodiment iPSC-TEC are administered together with activators of NK cells. Known NK activators useful for the practice of the invention include interferon alpha [74-76], interferon gamma [77], OK-432 [78-88]. The importance of NK activation can be seen in numerous publications which guide one of skill in the art on the practice of the invention. For example, a clinical trial demonstrated that patients who possess elevated levels of natural killer cell inhibitory proteins (soluble NKG2D ligands) demonstrated lower responses to checkpoint inhibitors [89]. Indeed, this should not be surprising since studies show that NK cell infiltration of tumors induces upregulation of antigen presentation in an interferon gamma associated manner, which renders tumor cells sensitive to T cell killing [90]. In one embodiment of the invention iPSC-TEC are utilized together with NK cell activators and checkpoint inhibitors. In other embodiments iPSC-TEC are combined with standard immunotherapies. In one embodiment said iPSC-TEC potently induce destruction of tumor blood vessels, wherein said damaged tumor blood vessels increase ability of immune cells to enter the tumor. Another example of the potency of combining immunotherapies is the example of Herceptin, in which approximately 1 out of 4 patients with the HER2neu antigen respond to treating. Interestingly it was found that lack of responsiveness correlates with inhibited NK cell activity [91-93]. Indeed, animal experiments demonstrate augmentation of Herceptin activity by stimulators of NK cells such as Poly (IC) and IL-12 [94, 95].

In one embodiment of the invention, iPSC-TEC are utilized together with macrophage activating means, and/or with activation M1 macrophage administration. Macrophages are key components of the innate immune system which play a principal role in the regulation of inflammation as well as physiological processes such as tissue remodeling [96, 97]. The diverse role of macrophages can be seen in conditions ranging from wound healing [98-101], to myocardial infarction [102-108], to renal failure [109-112] and liver failure [113].

Differentiated macrophages and their precursors are versatile cells that can adapt to micro environmental signals by altering their phenotype and function [114]. Although they have been studied for many years, it has only recently been shown that these cells comprise distinct sub-populations, known as classical M1 and alternative M2 [115]. Minoring the nomenclature of Th1 cells, M1 macrophages are described as the pro-inflammatory sub-type of macrophages induced by IFN-.gamma. and LPS. They produce effector molecules (e.g., reactive oxygen species) and pro-inflammatory cytokines (e.g., IL-12, TNF-.alpha. and IL-6) and they trigger Th1 polarized responses [116]. Macrophages can play a tumor inhibitory, as well as a tumor stimulatory role. Initial studies supported the role of macrophages in mediating antibody dependent cellular cytotoxicity in tumors [117-124], and thus being associated with potentiation of antitumor immune responses. Macrophages also possess the ability to directly recognize tumors by virtue of tumor expressed “eat-me” signals, which include the stress associated protein calreticulin [125, 126], which binds to the low-density lipoprotein receptor-related protein (LRP) on macrophages to induce phagocytosis [127]. Tumors protect themselves by expression of CD47, which binds to macrophage SIRP-1 and transduces an inhibitory signal [128]. Blockade of CD47 using antibodies results in remission of cancers mediated by macrophage activation [129-133]. Thus on the one hand, macrophages play an important role in induction of antitumor immunity. This can also be exemplified by some studies, involving administration of GM-CSF in order to augment macrophage numbers and activity in cancer patients [134-137].

Unfortunately, there is also evidence that macrophages support tumor growth. Accordingly, for the practice of the invention, care must be taken to inhibit the tumor promoting activities of macrophages. Studies in the osteoporotic mice strain, which lacks mature macrophages, demonstrate that tumors actually grow slower in animals deficient in macrophages [138]. Several other animal models have elegantly demonstrated that macrophages contribute to tumor growth, in part through stimulating on the angiogenic switch [139-141]. Numerous tumor biopsy studies have shown that there is a negative correlation between macrophage infiltration into tumors and patient survival [142-146]. The duality of macrophages in growth of tumors may be seen in studies of “inverse hormesis” in which low concentrations of antibodies targeting the tumor specific marker sialic acid N-glycolyl-neuraminic acid (Neu5Gc) actually leads to enhanced tumor growth in a macrophage dependent manner [147].

The importance of macrophages in clinical implementation of cancer therapeutics can be seen from results of a double blind clinical trials in metastatic colorectal cancer patients where cetuximab (anti-epidermal growth factor receptor (EGFR) monoclonal antibody (mAb)) was added to a protocol comprising of bevacizumab and chemotherapy. The addition of cetuximab actually resulted in decreased survival. In a study examining whether monocyte conversion to M2 angiogenic macrophages was responsible, investigators observed that CD163-positive M2 macrophages where found in high concentrations within the tumors of patients with colorectal carcinomas. These M2 cells expressed abundant levels of Fc-gamma receptors (FcγR) and PD-L1. Additionally, consistent with the M2 phenotype the cells generated large amounts of the immunosuppressive molecule IL-10 and the angiogenic mediator VEGF. When M2 cells were cultured with EGFR-positive tumor cells loaded with low concentrations of cetuximab, further augmentation of IL-10 and VEGF production was observed. These data suggest that under certain contexts, tumors manipulate macrophages to take on the M2 phenotype, and this subsequently leads to enhanced tumor progressing factors when tumor cells are bound by antibodies [148].

Manipulation of macrophages to inhibit M2 and shift to M1 phenotype may be performed using a variety of means. One theme that seems unifying is the ability of toll like receptor (TLR) agonists to influence this. In addition to cytokine differences, macrophages capable of killing tumor cells are usually known to express low levels of the inhibitory Fc gamma receptor IIb, whereas tumor promoting macrophages have high levels of this receptor [149]. Furthermore, tumor associated cytokines such as IL-4 and IL-10 are known to induce upregulation of the Fc gamma receptor IIB [150-153].

In one embodiment of the invention, iPSC-TEC are utilized together with agents and means that activate T cell responses. In one embodiment said T cell responses are utilized to kill tumor endothelial associated molecules. In other embodiments iPSC-TEC are utilized to enhance therapeutic ability of tumor infiltrating T cells. It is known that T cells are immune effectors against tumors, possessing ability to directly kill via CD8 cytotoxic cells [154-156], or indirectly killing tumors by activation of macrophages through interferon gamma production [157-159]. Additionally, T cells have been shown to convert pro-tumor M2 macrophages to M1 [160]. The importance of T cells in cancer is illustrated by positive correlation between tumor infiltrating lymphocytes and patient survival [161-165]. In addition, positive correlations between responses to various immunotherapies has been made with tumor infiltrating lymphocyte density [166, 167]. Increased T cell activity is associated with reduction in T regulatory (Treg) cells. Studies show that agents that cause suppression of Treg cells correlates with improved tumor control. Agents that inhibit Treg cells, which can be utilized together with iPSC-TEC include arsenic trioxide [168], cyclophosphamide [169-171], triptolide [170], gemcitabine [172], and artemether [173].

In one embodiment of the invention, Angiogenesis, the outgrowth of new blood vessels from pre-existing capillaries and post-capillary venules, occurs during embryonic development, in the uterus during the menstrual cycle, in the process of wound healing, and in pathological conditions [174]. In healthy adults, endothelial cells can maintain a quiescent state for years, whereas they proliferate and migrate to form new vessels in response to inflammatory conditions and during tumor growth. Studies have estimated as much as 30-40 fold more rapid proliferation of endothelial cells in tumors vs. normal vasculature [175-177]. Based on estimates that tumors fail to grow beyond 1-2 mm in the absence of new capillary growth, Dr. Judah Folkman put forth the central hypothesis that tumors release diffusible factors that stimulate endothelial cell proliferation in host capillary blood vessels [178]. Indeed, it has been estimated that eradication of one endothelial cell is capable of neutralizing of up to 100-300 tumor cells [179]. Since the immune system is in direct contact with the tumor vasculature, vaccination against endothelium is theoretically very promising for breaching the barriers created by the tumor microenvironment.

The goal in vaccination strategies is to raise immunity against antigens present in tumor endothelium while avoiding antigens that cross-react with healthy vasculature, thereby preventing deleterious autoimmune reactions. Since the landmark publication by Dr. Folkman, a catalog of molecular players involved in the process of tumor angiogenesis have been identified and characterized. Clinical outcomes of traditional anti-angiogenic therapies such as monoclonal antibodies have improved patient survival rates only modestly [180]. Vaccination against endothelial cells is poised to overcome the existing problems of drug resistance and adverse side effects associated with other approaches. This report reviews vaccination strategies against the tumor endothelium that have been tested to date, including DNA, protein and peptide vaccines using tumor-endothelium-associated antigens, as well as polyvalent vaccines comprising whole endothelial cells. Very encouraging data point toward the efficacy of vaccination in raising humoral and cell-mediated immunity against angiogenesis-associated antigens in cancer. Numerous approaches have been developed in attempts to selectively block tumor angiogenesis or induce collapse of tumor-associated blood vessels. While initial attempts such as development of endogenous inhibitors such as angiostatin and endostatin have failed, immunological means such as passive antibodies to VEGF (Avastin) have had success in terms of regulatory clearance and marketing approval. Drawbacks of Avastin include cardiotoxicity, development of resistance, has well as relatively poor survival advantage. Conceptually a more appealing method of inducing angiogenesis blockade would involve active immunization against several tumor endothelial associated antigens in the form of a polyvalent vaccine.

One major question that arises during attempts to induce active immunity to tumor associated endothelial is the “horror autotoxicus” potential of stimulating immunity towards non-malignant endothelium. We recently reviewed numerous works in which immunization to proliferating endothelial cells, whether syngeneic, allogeneic or xenogeneic results in selectivity of killing of tumors without damage to non-malignant tissues [181]. This is a fundamental point because numerous antigens found on tumor endothelial cells are also found on non-malignant cells, for example VEGFR is known to be associated with hematopoietic stem cell self-renewal. Despite this, as reviewed, immunization with VEGFR protein or plasmid does not result in ablation of hematopoietic stem cells, as would be expected. Accordingly multiple mechanisms must be at operation that discriminate tumor endothelial from cells expressing similar markers but are not under immunological attack and destruction as a result of the immunization. This is supported by clinical data in which immunization with HUVEC cells multiple times did not result in hematopoietic or other toxicities. While numerous attempts have been made at immunizing tumor bearing mice to endothelial antigens, the mechanistic data behind development of immunity has not been elucidated. Wei et al, for example, demonstrated involvement of antibodies targeting alpha V beta III integrin in suppression of tumor angiogenesis following immunization of mice with xenogeneic HUVEC cells [182]. Other studies have implicated T cell responses [183-185]. Indeed, in some situations not only collaboration between T cells and B cells is required for successful antitumor immunity, but also epitope spreading is observed between initial immunity towards tumor endothelium, which is subsequently followed by immunity towards tumor antigens themselves. Thus there is a high degree of variability of biological mechanisms between different active immunotherapies which target the tumor vasculature.

In some embodiments of the invention, human placental endothelial cell derived cellular vaccine product is used as a substitute for iPSC-TEC for vaccination. A similar vaccine has demonstrated human safety in an initial pilot clinical trial [186], as well as being shown to effectively reduce tumor growth in lung cancer, melanoma, and breast cancer [187]. In contrast to other endothelium based vaccines. Generating cancer endothelial targeting immunotherapies using placenta as a source of tissue has the unique properties of: a) Large donor supply. Since the vaccine is generated from placental endothelial cells, there exists a virtually unlimited supply of placentas, and additionally, each placenta is capable of generating a large number of doses; b) The vaccine is optimized for immunogenicity by pre-treatment with stimulators of HLA and CD80/86; and c) Placental endothelial is biologically naive, thus allowing for a higher degree of plasticity. The enhanced plasticity allows for higher levels of surface marker manipulation subsequent to treatment with cytokines.

For generation of immunogenic cells, iPSC cells may be differentiated into cells that resemble endothelial cells by culture in conditions such as certain growth factors are particularly important for the differentiation of pluripotent cells which have been maintained under defined conditions. In certain embodiments, pluripotent cells may be sequentially exposed to several defined media to promote differentiation into hematopoietic precursor cells. After culture and maintenance of the pluripotent cells in an essentially undifferentiated state in a first defined media (e.g., in a TeSR media), the cells may be exposed to a second defined media containing no or essentially no BMP4, VEGF, IL-3, Flt3 ligand, or GMCSF. The cells may then be exposed to a third defined media comprising BMP4, VEGF, IL-3, Flt3 ligand, and GMCSF to promote hematopoietic differentiation; alternately, the cells may be exposed to a third defined media comprising BMP4 and VEGF, and optionally FGF-2; followed by exposure to a fourth media comprising IL-3, Flt3 ligand, and GMCSF. The inventor has discovered that sequential exposure to a third defined media comprising BMP4 and VEGF, followed by exposure to a fourth media comprising IL-3, Flt3 ligand, and GMCSF can surprisingly result in substantial increases in the generation of hematopoietic precursor cells. As shown in the below examples, inclusion of FGF-2 in the third defined media resulted in a surprising increase in the differentiation of pluripotent cells into hematopoietic precursor cells. It has also been discovered that hypoxic conditions (e.g., exposure to an atmospheric pressure 5% O.sub.2), at least partial reaggregation of cells (e.g., using trypsin or TrypLE™), and/or formation of aggregates using defined ranges of cells in the formation of embryoid bodies (e.g., from about 200-1000 cells per aggregate) can also be used to further promote differentiation into hematopoietic precursor cells. Endothelial cells may be generated, for example, using the following protocol for implantation into an animal or human subject. Human cell-derived CD31+ cells may be cultured in either EGM™-2 medium (Lonza, Switzerland) or differentiation medium with 50 ng/mL rhVEGF and 5 ng/mL rhFGF-2 for 7 to 10 days. For further expansion and differentiation of endothelial cells, isolated CD31+ cells may be cultured in endothelial differentiation medium (Lonza catlalog #CC3202) containing VEGF, FGF, EGF, IGF, ascorbic acid and FBS or differentiation medium containing 50 ng/ml, vasular endothelial growth factor (VEGF), and FGF-2 (e.g., 50 ng/ml zebrafish FGF-2). Cells may be cultured for 2 to 3 weeks.] The following protocol may be used to induce endothelial differentiation. For expansion and differentiation of endothelial cells, isolated CD34+ cells may be seeded on gelatin-coated wells (1.5 to 2.times.10.sup.4 cells/cm.sup.2) in EGM-2MV medium (Cambrex). Collagen I-coated wells (BD labware) may also be used, although gelatin is typically less expensive than collagen I-coated wells. CD34+ cells may be cultured in hESC differentiation medium containing the endothelial growth factors, hVEGF.sub.165 (50 ng/mL) and FGF-2 (5 ng/mL). After about 7-10 days of incubation, the adherent cells may be harvested by trypsin-treatment and used for analyses. Endothelial cells may be evaluated by imaging a Matrigel plug. For example, the following protocol may be used to image cells: endothelial cells (ECs) derived from hESCs or iPSCs may be suspended in Matrigel (e.g., about 1 million cells in 1 ml) and injected subcutaneously into SCID Beige mice. FITC Dextran can then be injected intravenously at about day 14 before the removal of the matrigel plug. The plugs may be harvested and subjected to imaging using fluorescence imaging techniques, and the plug may be analyzed for the presence or absence of neovascularization.

Classical studies have shown that tumor infiltrating lymphocytes correlate with positive prognosis in various tumors [188-200]. Unfortunately, there are several important factors that prevent efficacy of infiltrating lymphocytes. Firstly, tumor masses originate from tumor stem cells, which possess distinctly different antigenic composition [201-205]. Accordingly, infiltration of lymphocytes, while useful for targeting tumor stem cell progeny, may not actually reach, or recognize tumor stem cells. This is also relevant in light of studies showing tumor stem cells possess various immune evasive molecules such as DAF, IL-10 and HLA-G. Secondly, tumors are known to possess high interstitial pressure, which physically limits ability of lymphocytes to enter the tumor mass, which often possesses necrotic tissue. Thirdly, tumor acidosis, hypoxia, and high adenosine concentrations have been demonstrated to selectively inhibit cytotoxic cells and promote T regulatory cells.

Despite theoretical obstacles to efficacy of immunotherapy of tumors, numerous studies have shown that in some patients, cancer vaccines, ranging from whole cell lysates of the 1970s to defined peptide vaccines, to nucleic acid vaccines, all induce in select patients some level of tumor regression. While in double blind trials these approaches often fail, due to heterogeneity of patients and tumors, in some patients documented durable responses are observed. It is not the scope of this paper to speculate on the heterogeneity, however, factors, which were not appreciated in previous studies, including polymorphisms in immune associated genes, expression of different mutations, diet, and gut microbiota content may all contribute observations of tumor eradication in some patients whereas in other patients no effect or even acceleration of tumor. Regardless of cause, it is documented that some patients highly respond to active vaccination against tumor antigens.

We propose that significantly higher killing of tumor cells may be achieved by directing antigen-specific immune responses toward the tumor endothelium. In contrast, to tumor stem cells, which are the desired target of an effective vaccination program, tumor endothelial cells are directly in contact with the circulatory system, thus permitting uninhibited access to immune cells and antibodies. Additionally, tumor endothelium is known to possess an increased level of prothrombotic molecules such as tissue factor [206-210]. Thus hypothetically, stimulation of thrombosis may be induced at a reduced threshold by immune cells/molecules targeting tumor angiogenesis as compared to existing vasculature in the body, which possesses numerous antithrombotic activities.

### Support for Targeting Tumor Endothelium

Early studies demonstrated that xenogeneic immunization with angiogenic proteins resulted in tumor regression. Breaking of self-tolerance using xenogeneic proteins is commonly used to elicit autoimmunity in models such as collagen induced arthritis and experimental autoimmune encephalitis (EAE). Accordingly, previous studies have shown that while inhibition of tolerance to self-proteins associated with oncogenic angiogenesis results in inhibition of tumor growth, alterations to physiological processes such as wound healing or menstruation where not observed. This perhaps indicates the ability of the immune system to differentiate between pathological conditions of angiogenesis versus physiological. One potential analogy are clinical studies using antigen specific T cells for autoimmunity as a vaccine. In these studies, despite T cells being administered in an immunogenic manner, only antigen specific, idiotypic T cells are immunologically attacked and not all T cells.

A fundamental question determining feasibility of vaccine-induced killing of tumor vasculature is whether antigens exist on the tumor endothelium that are not expressed on physiologically normal blood vessels, and whether immunity could be raised against such antigens. The roundabout receptor (ROBO)-4 is a transmembrane protein that was originally found to orchestrate the neuronal guidance mechanism of the nervous system [211]. ROBO4 was found to be selectively expressed on tumor endothelial cells but not healthy vasculature [212]. Zhuang et al demonstrated that mice immunized with the extracellular domain of mouse Robo4, showed a strong antibody response to Robo4, with no objectively detectable adverse effects on health, including normal menstruation and wound healing. Robo4 vaccinated mice showed impaired fibrovascular invasion and angiogenesis in a rodent sponge implantation assay, as well as a reduced growth of implanted syngeneic Lewis lung carcinoma. The anti-tumor effect of Robo4 vaccination was present in CD8 deficient mice but absent in B cell or IgG1 knockout mice, suggesting antibody dependent cell mediated cytotoxicity (ADCC) as the anti-vascular/anti-tumor mechanism [213]. Another antigen that is more ubiquitously found throughout the body, but with higher expression on tumor endothelial cells is the VEGF receptor 2 (VEGFR2) which is typically found on hematopoietic stem cells and endothelial progenitor cells [11, 12, 214-217]. Despite expression on non-malignant tissue, successful induction of antitumor immunity has been demonstrated using various immunization means against this antigen. Yan et al utilized irradiated AdVEGFR2-infected cell vaccine-based immunotherapy in the weakly immunogenic and highly metastatic 4T1 murine mammary cancer model. Lethally irradiated, virus-infected 4T1 cells were used as vaccines. Vaccination with lethally irradiated AdVEGFR2-infected 4T1 cells inhibited subsequent tumor growth and pulmonary metastasis compared with challenge inoculations. Angiogenesis was inhibited, and the number of CD8+ T lymphocytes was increased within the tumors. Antitumor activity was also caused by the adoptive transfer of isolated spleen lymphocytes, thus demonstrating induction of tumor specific immunity [218]. Other approaches have been utilized to induce immunity to VEGFR2, which resulted in induction of tumor regression without systemic toxicities [219-224]. Other approaches have been utilized to induce immunity to VEGFR2, which resulted in induction of tumor regression without systemic toxicities [219-224]. Tumor endothelial marker 1 or endosialin is another antigen found selectively on the tumor vasculature. Facciponte et al demonstrated that a DNA vaccination targeting endosialin reduced tumor vascularity, increased CD3+ T cell infiltration, and was correlated with significant inhibition of tumor growth. Epitope spreading to tumor antigens following the initial immune response against the tumor vasculature gives evidence that targeting the tumor endothelium may activate a cascade of pathways conducive to tumor regression. Additionally, the DNA vaccination against endosialin did not affect other angiogenesis dependent physiological processes, exhibiting no adverse effects on menstruation, embryonic development, pregnancy, and wound healing in mouse models [225]. Other markers associated with tumor blood vessels have been utilized therapeutically in animal models for vaccination purposes including survivin [226-228], endosialin [229], and xenogeneic FGF2R [230], VEGF [231], VEGF-R2 [232], MMP-2 [233], and endoglin [234, 235].

### EXAMPLE

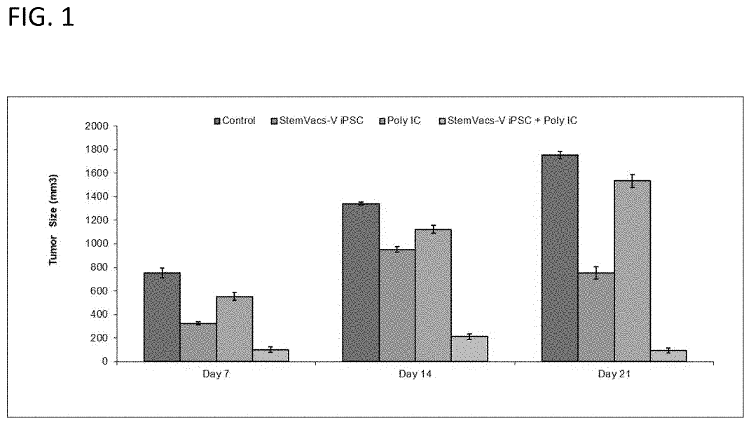

iPSC expressing α-Gal epitope (Galα1,3Galα1,4GlcNAc-R) were differentiated into tumor endothelial like cells by culture in VEGF (10 ng/ml), PDGF-BB (5 ng/ml), PGE-2 (100 pg/ml). Cells were administered alone, 500,000 per mouse or together with Poly IC (5 ng/mouse) into mice bearing B16 melanoma. The results are shown in FIG. 1.

### REFERENCES

1. Kerbel, R. S., Reappraising antiangiogenic therapy for breast cancer. Breast, 2011. 20 Suppl 3: p. S56-60.
2. Kerbel, R. S., Issues regarding improving the impact of antiangiogenic drugs for the treatment of breast cancer. Breast, 2009. 18 Suppl 3: p. S41-7.
3. Lu, K. V. and G. Bergers, Mechanisms of evasive resistance to anti-VEGF therapy in glioblastoma. CNS Oncol, 2013. 2(1): p. 49-65.
4. Chen, J., et al., Severe Cardiotoxicity in a Patient with Colorectal Cancer Treated with Bevacizumab. Anticancer Res, 2017. 37(8): p. 4557-4561.
5. Economopoulou, P., et al., Cancer therapy and cardiovascular risk: focus on bevacizumab. Cancer Manag Res, 2015. 7: p. 133-43.
6. des Guetz, G., et al., Cardiovascular toxicity of anti-angiogenic drugs. Target Oncol, 2011. 6(4): p. 197-202.
7. Advani, R. H., et al., Cardiac toxicity associated with bevacizumab (Avastin) in combination with CHOP chemotherapy for peripheral T cell lymphoma in ECOG 2404 trial. Leuk Lymphoma, 2012. 53(4): p. 718-20.
8. Hawkes, E. A., et al., Cardiotoxicity in patients treated with bevacizumab is potentially reversible. J Clin Oncol, 2011. 29(18): p. e560-2.
9. Yardley, D. A., Integrating bevacizumab into the treatment of patients with early-stage breast cancer: focus on cardiac safety. Clin Breast Cancer, 2010. 10(2): p. 119-29.
10. Drimal, J., et al., Cardiovascular toxicity of the first line cancer chemotherapeutic agents: doxorubicin, cyclophosphamide, streptozotocin and bevacizumab. Neuro Endocrinol Lett, 2006. 27 Suppl 2: p. 176-9.
11. Rafii, S., et al., Angiogenic factors reconstitute hematopoiesis by recruiting stem cells from bone marrow microenvironment. Ann N Y Acad Sci, 2003. 996: p. 49-60.
12. Hattori, K., et al., Placental growth factor reconstitutes hematopoiesis by recruiting VEGFR1(+) stem cells from bone-marrow microenvironment. Nat Med, 2002. 8(8): p. 841-9.
13. DiNitto, J. P. and J. C. Wu, Molecular mechanisms of drug resistance in tyrosine kinases cAbl and cKit. Crit Rev Biochem Mol Biol, 2011. 46(4): p. 295-309.
14. Cao, Y., et al., Forty-year journey of angiogenesis translational research. Sci Transl Med, 2011. 3(114): p. 114rv3.
15. Abdollahi, A. and J. Folkman, Evading tumor evasion: current concepts and perspectives of anti-angiogenic cancer therapy. Drug Resist Updat, 2010. 13(1-2): p. 16-28.
16. Chen, H. X., R. E. Gore-Langton, and B. D. Cheson, Clinical trials referral resource: Current clinical trials of the anti-VEGF monoclonal antibody bevacizumab. Oncology (Williston Park), 2001. 15(8): p. 1017, 1020, 1023-6.
17. Krupitskaya, Y. and H. A. Wakelee, Ramucirumab, a fully human mAb to the transmembrane signaling tyrosine kinase VEGFR-2 for the potential treatment of cancer. Curr Opin Investig Drugs, 2009. 10(6): p. 597-605.
18. Abrams, T. J., et al., SU11248 inhibits KIT and platelet-derived growth factor receptor beta in preclinical models of human small cell lung cancer. Mol Cancer Ther, 2003. 2(5): p. 471-8.
19. Carlisle, B., et al., Benefit, Risk, and Outcomes in Drug Development: A Systematic Review of Sunitinib. J Natl Cancer Inst, 2016. 108(1).
20. Izzedine, H., et al., Sunitinib malate. Cancer Chemother Pharmacol, 2007. 60(3): p. 357-64.
21. Viola, D., V. Cappagli, and R. Elisei, Cabozantinib (XL184) for the treatment of locally advanced or metastatic progressive medullary thyroid cancer. Future Oncol, 2013. 9(8): p. 1083-92.
22. Roy, S., et al., A novel multiple tyrosine-kinase targeted agent to explore the future perspectives of anti-angiogenic therapy for the treatment of multiple solid tumors: cabozantinib. Anticancer Agents Med Chem, 2015. 15(1): p. 37-47.
23. Tannir, N. M., G. Schwab, and V. Grunwald, Cabozantinib: an Active Novel Multikinase Inhibitor in Renal Cell Carcinoma. Curr Oncol Rep, 2017. 19(2): p. 14.
24. Yakes, F. M., et al., Cabozantinib (XL184), a novel MET and VEGFR2 inhibitor, simultaneously suppresses metastasis, angiogenesis, and tumor growth. Mol Cancer Ther, 2011. 10(12): p. 2298-308.
25. Gril, B., et al., The B-Raf status of tumor cells may be a significant determinant of both antitumor and anti-angiogenic effects of pazopanib in xenograft tumor models. PLoS One, 2011. 6(10): p. e25625.
26. van Geel, R. M., J. H. Beijnen, and J. H. Schellens, Concise drug review: pazopanib and axitinib. Oncologist, 2012. 17(8): p. 1081-9.
27. Ferrero, S., et al., Pharmacokinetic drug evaluation of pazopanib for the treatment of uterine leiomyosarcomas. Expert Opin Drug Metab Toxicol, 2017. 13(8): p. 881-889.
28. Gaumann, A. K., et al., Receptor tyrosine kinase inhibitors: Are they real tumor killers? Int J Cancer, 2016. 138(3): p. 540-54.
29. Miura, K., et al., The preclinical development of regorafenib for the treatment of colorectal cancer. Expert Opin Drug Discov, 2014. 9(9): p. 1087-101.
30. Rimassa, L., et al., Regorafenib for the treatment of unresectable hepatocellular carcinoma. Expert Rev Anticancer Ther, 2017. 17(7): p. 567-576.
31. Ohga, N., et al., Heterogeneity of tumor endothelial cells: comparison between tumor endothelial cells isolated from high- and low-metastatic tumors. Am J Pathol, 2012. 180(3): p. 1294-307.
32. Akiyama, K., et al., Tumor endothelial cells acquire drug resistance by MDR1 up-regulation via VEGF signaling in tumor microenvironment. Am J Pathol, 2012. 180(3): p. 1283-93.
33. Ohmura-Kakutani, H., et al., Identification of tumor endothelial cells with high aldehyde dehydrogenase activity and a highly angiogenic phenotype. PLoS One, 2014. 9(12): p. e113910.
34. Sruthi, T. V., et al., Horizontal transfer of miR-23a from hypoxic tumor cell colonies can induce angiogenesis. J Cell Physiol, 2017.
35. Kosaka, N., et al., Neutral sphingomyelinase 2 (nSMase2)-dependent exosomal transfer of angiogenic microRNAs regulate cancer cell metastasis. J Biol Chem, 2013. 288(15): p. 10849-59.
36. Zhang, Y., et al., Tumacrophage: macrophages transformed into tumor stem-like cells by virulent genetic material from tumor cells. Oncotarget, 2017. 8(47): p. 82326-82343.
37. Abdouh, M., et al., Exosomes isolated from cancer patients' sera transfer malignant traits and confer the same phenotype of primary tumors to oncosuppressor-mutated cells. J Exp Clin Cancer Res, 2017. 36(1): p. 113.
38. Semina, S. E., et al., Horizontal Transfer of Tamoxifen Resistance in MCF-7 Cell Derivates: Proteome Study. Cancer Invest, 2017. 35(8): p. 506-518.
39. Cai, J., et al., Functional transferred DNA within extracellular vesicles. Exp Cell Res, 2016. 349(1): p. 179-183.
40. Hamam, D., et al., Transfer of malignant trait to BRCA1 deficient human fibroblasts following exposure to serum of cancer patients. J Exp Clin Cancer Res, 2016. 35: p. 80.
41. Berridge, M. V., L. Dong, and J. Neuzil, Mitochondrial DNA in Tumor Initiation, Progression, and Metastasis: Role of Horizontal mtDNA Transfer. Cancer Res, 2015. 75(16): p. 3203-8.
42. Chen, W. X., et al., Exosomes from drug-resistant breast cancer cells transmit chemoresistance by a horizontal transfer of microRNAs. PLoS One, 2014. 9(4): p. e95240.
43. Zhu, X., et al., BCR-ABL1-positive microvesicles transform normal hematopoietic transplants through genomic instability: implications for donor cell leukemia. Leukemia, 2014. 28(8): p. 1666-75.
44. Stein, J., et al., Origin of leukemic relapse after bone marrow transplantation: comparison of cytogenetic and molecular analyses. Blood, 1989. 73(7): p. 2033-40.
45. Angara, K., T. F. Bonin, and A. S. Arbab, Vascular Mimicry: A Novel Neovascularization Mechanism Driving Anti-Angiogenic Therapy (AAT) Resistance in Glioblastoma. Transl Oncol, 2017. 10(4): p. 650-660.
46. Sood, A. K., et al., The clinical significance of tumor cell-lined vasculature in ovarian carcinoma: implications for anti-vasculogenic therapy. Cancer Biol Ther, 2002. 1(6): p. 661-4.
47. Mahase, S., et al., Hypoxia-Mediated Mechanisms Associated with Antiangiogenic Treatment Resistance in Glioblastomas. Am J Pathol, 2017. 187(5): p. 940-953.
48. Pinto, M. P., et al., Escaping Antiangiogenic Therapy: Strategies Employed by Cancer Cells. Int J Mol Sci, 2016. 17(9).
49. Schnegg, C. I., et al., Induction of Vasculogenic Mimicry Overrides VEGF-A Silencing and Enriches Stem-like Cancer Cells in Melanoma. Cancer Res, 2015. 75(8): p. 1682-90.
50. Soda, Y., et al., Transdifferentiation of glioblastoma cells into vascular endothelial cells. Proc Natl Acad Sci USA, 2011. 108(11): p. 4274-80.
51. Li, Q., et al., Angiogenesis inhibitors for the treatment of small cell lung cancer (SCLC): A meta-analysis of 7 randomized controlled trials. Medicine (Baltimore), 2017. 96(13): p. e6412.
52. Lombardi, G., et al., Effectiveness of antiangiogenic drugs in glioblastoma patients: A systematic review and meta-analysis of randomized clinical trials. Crit Rev Oncol Hematol, 2017. 111: p. 94-102.
53. Roviello, G., et al., The role of bevacizumab in solid tumours: A literature based meta-analysis of randomised trials. Eur J Cancer, 2017. 75: p. 245-258.
54. Shih, T. and C. Lindley, Bevacizumab: an angiogenesis inhibitor for the treatment of solid malignancies. Clin Ther, 2006. 28(11): p. 1779-802.
55. Chase, J. L., Clinical use of anti-vascular endothelial growth factor monoclonal antibodies in metastatic colorectal cancer. Pharmacotherapy, 2008. 28(11 Pt 2): p. 23S-30S.
56. Yu, J., et al., Efficacy and safety of angiogenesis inhibitors in advanced gastric cancer. a systematic review and meta-analysis. J Hematol Oncol, 2016. 9(1): p. 111.
57. Ciliberto, D., et al., A systematic review and meta-analysis of randomized trials on the role of targeted therapy in the management of advanced gastric cancer: Evidence does not translate? Cancer Biol Ther, 2015. 16(8): p. 1148-59.
58. Welsh, S. J. and K. Fife, Pazopanib for the treatment of renal cell carcinoma. Future Oncol, 2015. 11(8): p. 1169-79.
59. Khan, K., D. Cunningham, and I. Chau, Targeting Angiogenic Pathways in Colorectal Cancer: Complexities, Challenges and Future Directions. Curr Drug Targets, 2017. 18(1): p. 56-71.
60. Iacovelli, R., et al., Inhibition of the VEGF/VEGFR pathway improves survival in advanced kidney cancer: a systematic review and meta-analysis. Curr Drug Targets, 2015. 16(2): p. 164-70.
61. Piperdi, B., A. Merla, and R. Perez-Soler, Targeting angiogenesis in squamous non-small cell lung cancer. Drugs, 2014. 74(4): p. 403-13.
62. von Baumgarten, L., et al., Bevacizumab has differential and dose-dependent effects on glioma blood vessels and tumor cells. Clin Cancer Res, 2011. 17(19): p. 6192-205.
63. Yuan, F., et al., Time-dependent vascular regression and permeability changes in established human tumor xenografts induced by an anti-vascular endothelial growth factor/vascular permeability factor antibody. Proc Natl Acad Sci USA, 1996. 93(25): p. 14765-70.
64. Aalders, K. C., et al., Anti-angiogenic treatment in breast cancer: Facts, successes, failures and future perspectives. Cancer Treat Rev, 2017. 53: p. 98-110.
65. Huang, M., et al., Macrophage inhibitory cytokine-1 induced by a high-fat diet promotes prostate cancer progression by stimulating tumor-promoting cytokine production from tumor stromal cells. Cancer Commun (Lond), 2021.
66. Bareche, Y., et al., High-dimensional analysis of the adenosine pathway in high-grade serous ovarian cancer. J Immunother Cancer, 2021. 9(3).
67. Martin-Fontecha, A., et al., Induced recruitment of NK cells to lymph nodes provides IFN-gamma for T(H)1 priming. Nat Immunol, 2004. 5(12): p. 1260-5.
68. Morandi, B., et al., NK cells of human secondary lymphoid tissues enhance T cell polarization via IFN-gamma secretion. Eur J Immunol, 2006. 36(9): p. 2394-400.
69. Ksienzyk, A., et al., IRF-1 expression is essential for natural killer cells to suppress metastasis. Cancer Res, 2011. 71(20): p. 6410-8.
70. Lopez-Soto, A., et al., Control of Metastasis by NK Cells. Cancer Cell, 2017. 32(2): p. 135-154.
71. Krasnova, Y., et al., Bench to bedside: NK cells and control of metastasis. Clin Immunol, 2017. 177: p. 50-59.
72. Putz, E. M., et al., NK cell heparanase controls tumor invasion and immune surveillance. J Clin Invest, 2017. 127(7): p. 2777-2788.
73. Morvan, M. G. and L. L. Lanier, NK cells and cancer: you can teach innate cells new tricks. Nat Rev Cancer, 2016. 16(1): p. 7-19.
74. Kozlowska, A., J. Mackiewicz, and A. Mackiewicz, Therapeutic gene modified cell based cancer vaccines. Gene, 2013. 525(2): p. 200-7.
75. Hervas-Stubbs, S., et al., Direct effects of type I interferons on cells of the immune system. Clin Cancer Res, 2011. 17(9): p. 2619-27.
76. Novick, D., et al., The neutralization of type I IFN biologic actions by anti-IFNAR-2 monoclonal antibodies is not entirely due to inhibition of Jak-Stat tyrosine phosphorylation. J Interferon Cytokine Res, 2000. 20(11): p. 971-82.
77. Gonsky, R., et al., Enhancer role of STAT5 in CD2 activation of IFN-gamma gene expression. J Immunol, 2004. 173(10): p. 6241-7.
78. Pan, K., et al., OK-432 synergizes with IFN-gamma to confer dendritic cells with enhanced antitumor immunity. Immunol Cell Biol, 2014. 92(3): p. 263-74.
79. Chen, I. J., et al., Vaccination with OK-432 followed by TC-1 tumor lysate leads to significant antitumor effects. Reprod Sci, 2011. 18(7): p. 687-94.
80. Takahashi, E., et al., Induction of CD16+ CD56bright NK cells with antitumour cytotoxicity not only from CD16- CD56bright NK Cells but also from CD16- CD56dim NK cells. Scand J Immunol, 2007. 65(2): p. 126-38.
81. Okinaga, K., et al., Effect of immunotherapy and spleen preservation on immunological function in patients with gastric cancer. J Exp Clin Cancer Res, 2006. 25(3): p. 339-49.
82. Sato, M., et al., Generation of mature dendritic cells fully capable of T helper type 1 polarization using OK-432 combined with prostaglandin E(2). Cancer Sci, 2003. 94(12): p. 1091-8.
83. Okamoto, M., et al., Enhancement of anti-tumor immunity by lipoteichoic acid-related molecule isolated from OK-432, a streptococcal agent, in athymic nude mice bearing human salivary adenocarcinoma: role of natural killer cells. Anticancer Res, 2002. 22(6A): p. 3229-39.
84. Uehara, K., et al., Systemic administration of liposome-encapsulated OK-432 prolongs the survival of rats with hepatocellular carcinoma through the induction of IFN-gamma-producing hepatic lymphocytes. J Gastroenterol Hepatol, 2002. 17(1): p. 81-90.
85. Okamoto, M., et al., Induction of Th1-type cytokines by lipoteichoic acid-related preparation isolated from OK-432, a penicillin-killed streptococcal agent. Immunopharmacology, 2000. 49(3): p. 363-76.
86. Yamagiwa, S., et al., Liposome-encapsulated OK-432 specifically and sustainedly induces hepatic natural killer cells and intermediate T cell receptor cells. J Gastroenterol Hepatol, 2000. 15(5): p. 542-9.
87. Yamamoto, K., et al., Effects of OK-432 on the proliferation and cytotoxicity of lymphokine-activated killer (LAK) cells. J Immunother, 1999. 22(1): p. 33-40.
88. Kurosawa, S., et al., The concurrent administration of OK432 augments the antitumor vaccination effect with tumor cells by sustaining locally infiltrating natural killer cells. Cancer Immunol Immunother, 1996. 43(1): p. 31-8.
89. Maccalli, C., et al., Soluble NKG2D ligands are biomarkers associated with the clinical outcome to immune checkpoint blockade therapy of metastatic melanoma patients. Oncoimmunology, 2017. 6(7): p. e1323618.
90. Goding, S. R., et al., Adoptive transfer of natural killer cells promotes the anti-tumor efficacy of T cells. Clin Immunol, 2017. 177: p. 76-86.
91. Muraro, E., et al., Improved Natural Killer cell activity and retained anti-tumor CD8(+) T cell responses contribute to the induction of a pathological complete response in HER2-positive breast cancer patients undergoing neoadjuvant chemotherapy. J Transl Med, 2015. 13: p. 204.
92. Lee, S. C., et al., Natural killer (NK): dendritic cell (DC) cross talk induced by therapeutic monoclonal antibody triggers tumor antigen-specific T cell immunity. Immunol Res, 2011. 50(2-3): p. 248-54.
93. Beano, A., et al., Correlation between NK function and response to trastuzumab in metastatic breast cancer patients. J Transl Med, 2008. 6: p. 25.
94. Jaime-Ramirez, A. C., et al., IL-12 enhances the antitumor actions of trastuzumab via NK cell IFN-gamma production. J Immunol, 2011. 186(6): p. 3401-9.
95. Charlebois, R., et al., PolyI:C and CpG Synergize with Anti-ErbB2 mAb for Treatment of Breast Tumors Resistant to Immune Checkpoint Inhibitors. Cancer Res, 2017. 77(2): p. 312-319.
96. van Furth, R. and Z. A. Cohn, The origin and kinetics of mononuclear phagocytes. J Exp Med, 1968. 128(3): p. 415-35.
97. Wynn, T. A., A. Chawla, and J. W. Pollard, Macrophage biology in development, homeostasis and disease. Nature, 2013. 496(7446): p. 445-55.
98. Smith, T. D., et al., Harnessing macrophage plasticity for tissue regeneration. Adv Drug Deliv Rev, 2017.
99. Vannella, K. M. and T. A. Wynn, Mechanisms of Organ Injury and Repair by Macrophages. Annu Rev Physiol, 2017. 79: p. 593-617.
100. Boddupalli, A., L. Zhu, and K. M. Bratlie, Methods for Implant Acceptance and Wound Healing: Material Selection and Implant Location Modulate Macrophage and Fibroblast Phenotypes. Adv Healthc Mater, 2016. 5(20): p. 2575-2594.
101. Snyder, R. J., et al., Macrophages: A review of their role in wound healing and their therapeutic use. Wound Repair Regen, 2016. 24(4): p. 613-29.
102. Gombozhapova, A., et al., Macrophage activation and polarization in post-infarction cardiac remodeling. J Biomed Sci, 2017. 24(1): p. 13.
103. Hu, Y., et al., Class A scavenger receptor attenuates myocardial infarction-induced cardiomyocyte necrosis through suppressing M1 macrophage subset polarization. Basic Res Cardiol, 2011. 106(6): p. 1311-28.
104. Ma, Y., et al., Matrix metalloproteinase-28 deletion exacerbates cardiac dysfunction and rupture after myocardial infarction in mice by inhibiting M2 macrophage activation. Circ Res, 2013. 112(4): p. 675-88.
105. Lee, C. W., et al., Macrophage heterogeneity of culprit coronary plaques in patients with acute myocardial infarction or stable angina. Am J Clin Pathol, 2013. 139(3): p. 317-22.
106. Yan, X., et al., Temporal dynamics of cardiac immune cell accumulation following acute myocardial infarction. J Mol Cell Cardiol, 2013. 62: p. 24-35.
107. Fernandez-Velasco, M., S. Gonzalez-Ramos, and L. Bosca, Involvement of monocytes/macrophages as key factors in the development and progression of cardiovascular diseases. Biochem J, 2014. 458(2): p. 187-93.
108. de Couto, G., et al., Macrophages mediate cardioprotective cellular postconditioning in acute myocardial infarction. J Clin Invest, 2015. 125(8): p. 3147-62.
109. Guiteras, R., M. Flaquer, and J. M. Cruzado, Macrophage in chronic kidney disease. Clin Kidney J, 2016. 9(6): p. 765-771.
110. Meng, X. M., et al., Macrophage Phenotype in Kidney Injury and Repair. Kidney Dis (Basel), 2015. 1(2): p. 138-46.
111. Yamamoto, S., et al., Atherosclerosis following renal injury is ameliorated by pioglitazone and losartan via macrophage phenotype. Atherosclerosis, 2015. 242(1): p. 56-64.
112. Li, C., et al., Enhanced M1 and Impaired M2 Macrophage Polarization and Reduced Mitochondrial Biogenesis via Inhibition of AMP Kinase in Chronic Kidney Disease. Cell Physiol Biochem, 2015. 36(1): p. 358-72.
113. Sun, Y. Y., et al., Macrophage Phenotype in Liver Injury and Repair. Scand J Immunol, 2017. 85(3): p. 166-174.
114. Gratchev, A., et al., Mphi1 and Mphi2 can be re-polarized by Th2 or Th1 cytokines, respectively, and respond to exogenous danger signals. Immunobiology, 2006. 211(6-8): p. 473-86.
115. Mills, C. D., M1 and M2 Macrophages: Oracles of Health and Disease. Crit Rev Immunol, 2012. 32(6): p. 463-88.
116. Mills, C. D. and K. Ley, M1 and M2 macrophages: the chicken and the egg of immunity. J Innate Immun, 2014. 6(6): p. 716-26.
117. Alsaid, H., et al., Non invasive imaging assessment of the biodistribution of GSK2849330, an ADCC and CDC optimized anti HER3 mAb, and its role in tumor macrophage recruitment in human tumor-bearing mice. PLoS One, 2017. 12(4): p. e0176075.
118. Josephs, D. H., et al., Anti-Folate Receptor-alpha IgE but not IgG Recruits Macrophages to Attack Tumors via TNFalpha/MCP-1 Signaling. Cancer Res, 2017. 77(5): p. 1127-1141.
119. Velmurugan, R., et al., Macrophage-Mediated Trogocytosis Leads to Death of Antibody-Opsonized Tumor Cells. Mol Cancer Ther, 2016. 15(8): p. 1879-89.
120. Gul, N. and M. van Egmond, Antibody-Dependent Phagocytosis of Tumor Cells by Macrophages: A Potent Effector Mechanism of Monoclonal Antibody Therapy of Cancer. Cancer Res, 2015. 75(23): p. 5008-13.
121. Church, A. K., et al., Anti-CD20 monoclonal antibody-dependent phagocytosis of chronic lymphocytic leukaemia cells by autologous macrophages. Clin Exp Immunol, 2016. 183(1): p. 90-101.
122. Shi, Y., et al., Trastuzumab triggers phagocytic killing of high HER2 cancer cells in vitro and in vivo by interaction with Fcgamma receptors on macrophages. J Immunol, 2015. 194(9): p. 4379-86.
123. Weiskopf, K. and I. L. Weissman, Macrophages are critical effectors of antibody therapies for cancer. MAbs, 2015. 7(2): p. 303-10.
124. Oflazoglu, E., et al., Macrophages contribute to the antitumor activity of the anti-CD30 antibody SGN-30. Blood, 2007. 110(13): p. 4370-2.
125. Osman, R., et al., Calreticulin Release at an Early Stage of Death Modulates the Clearance by Macrophages of Apoptotic Cells. Front Immunol, 2017. 8: p. 1034.
126. Feng, M., et al., Macrophages eat cancer cells using their own calreticulin as a guide: roles of TLR and Btk. Proc Natl Acad Sci USA, 2015. 112(7): p. 2145-50.
127. Chao, M. P., et al., Calreticulin is the dominant pro-phagocytic signal on multiple human cancers and is counterbalanced by CD47. Sci Transl Med, 2010. 2(63): p. 63ra94.
128. Murata, Y., et al., The CD47-SIRPalpha signaling system: its physiological roles and therapeutic application. J Biochem, 2014. 155(6): p. 335-44.
129. Roberts, D. D., S. Kaur, and D. R. Soto-Pantoja, Therapeutic targeting of the thrombospondin-1 receptor CD47 to treat liver cancer. J Cell Commun Signal, 2015. 9(1): p. 101-2.
130. Liu, J., et al., Pre-Clinical Development of a Humanized Anti-CD47 Antibody with Anti-Cancer Therapeutic Potential. PLoS One, 2015. 10(9): p. e0137345.
131. Weiskopf, K., et al., CD47-blocking immunotherapies stimulate macrophage-mediated destruction of small-cell lung cancer. J Clin Invest, 2016. 126(7): p. 2610-20.
132. Weiskopf, K., et al., Eradication of Canine Diffuse Large B-Cell Lymphoma in a Murine Xenograft Model with CD47 Blockade and Anti-CD20. Cancer Immunol Res, 2016. 4(12): p. 1072-1087.
133. Zeng, D., et al., A fully human anti-CD47 blocking antibody with therapeutic potential for cancer. Oncotarget, 2016. 7(50): p. 83040-83050.
134. Liljefors, M., et al., Influence of varying doses of granulocyte-macrophage colony-stimulating factor on pharmacokinetics and antibody-dependent cellular cytotoxicity. Cancer Immunol Immunother, 2008. 57(3): p. 379-88.
135. Tarr, P. E., Granulocyte-macrophage colony-stimulating factor and the immune system. Med Oncol, 1996. 13(3): p. 133-40.
136. Ragnhammar, P., et al., Cytotoxicity of white blood cells activated by granulocyte-colony-stimulating factor, granulocyte/macrophage-colony-stimulating factor and macrophage-colony-stimulating factor against tumor cells in the presence of various monoclonal antibodies. Cancer Immunol Immunother, 1994. 39(4): p. 254-62.
137. Ragnhammar, P., Anti-tumoral effect of GM-CSF with or without cytokines and monoclonal antibodies in solid tumors. Med Oncol, 1996. 13(3): p. 167-76.
138. Lin, E. Y., et al., Colony-stimulating factor 1 promotes progression of mammary tumors to malignancy. J Exp Med, 2001. 193(6): p. 727-40.
139. Aharinejad, S., et al., Colony-stimulating factor-1 blockade by antisense oligonucleotides and small interfering RNAs suppresses growth of human mammary tumor xenografts in mice. Cancer Res, 2004. 64(15): p. 5378-84.
140. Lin, E. Y., et al., Macrophages regulate the angiogenic switch in a mouse model of breast cancer. Cancer Res, 2006. 66(23): p. 11238-46.
141. Lin, E. Y. and J. W. Pollard, Tumor-associated macrophages press the angiogenic switch in breast cancer. Cancer Res, 2007. 67(11): p. 5064-6.
142. Zhang, W. J., et al., Hypoxia-inducible factor-1 alpha Correlates with Tumor-Associated Macrophages Infiltration, Influences Survival of Gastric Cancer Patients. J Cancer, 2017. 8(10): p. 1818-1825.
143. Yuan, X., et al., Prognostic significance of tumor-associated macrophages in ovarian cancer: A meta-analysis. Gynecol Oncol, 2017. 147(1): p. 181-187.
144. Ma, C., et al., CD163-positive cancer cells are potentially associated with high malignant potential in clear cell renal cell carcinoma. Med Mol Morphol, 2017.
145. Shi, Y., et al., Tumour-associated macrophages secrete pleiotrophin to promote PTPRZ1 signalling in glioblastoma stem cells for tumour growth. Nat Commun, 2017. 8: p. 15080.
146. Zhao, X., et al., Prognostic significance of tumor-associated macrophages in breast cancer: a meta-analysis of the literature. Oncotarget, 2017. 8(18): p. 30576-30586.
147. Pearce, O. M., et al., Inverse hormesis of cancer growth mediated by narrow ranges of tumor-directed antibodies. Proc Natl Acad Sci USA, 2014. 111(16): p. 5998-6003.
148. Pander, J., et al., Activation of tumor-promoting type 2 macrophages by EGFR-targeting antibody cetuximab. Clin Cancer Res, 2011. 17(17): p. 5668-73.
149. Clynes, R. A., et al., Inhibitory Fc receptors modulate in vivo cytotoxicity against tumor targets. Nat Med, 2000. 6(4): p. 443-6.
150. Pricop, L., et al., Differential modulation of stimulatory and inhibitory Fc gamma receptors on human monocytes by Th1 and Th2 cytokines. J Immunol, 2001. 166(1): p. 531-7.
151. Tridandapani, S., et al., Regulated expression and inhibitory function of Fcgamma RIIB in human monocytic cells. J Biol Chem, 2002. 277(7): p. 5082-9.
152. Joshi, T., et al., Molecular analysis of expression and function of hFcgammaRIIbl and b2 isoforms in myeloid cells. Mol Immunol, 2006. 43(7): p. 839-50.
153. Wijngaarden, S., et al., A shift in the balance of inhibitory and activating Fcgamma receptors on monocytes toward the inhibitory Fcgamma receptor IIb is associated with prevention of monocyte activation in rheumatoid arthritis. Arthritis Rheum, 2004. 50(12): p. 3878-87.
154. Zumwalt, T. J., et al., Active secretion of CXCL10 and CCL5 from colorectal cancer microenvironments associates with GranzymeB+ CD8+ T-cell infiltration. Oncotarget, 2015. 6(5): p. 2981-91.
155. Ochsenbein, A. F., Principles of tumor immunosurveillance and implications for immunotherapy. Cancer Gene Ther, 2002. 9(12): p. 1043-55.
156. Ochsenbein, A. F., et al., Roles of tumour localization, second signals and cross priming in cytotoxic T-cell induction. Nature, 2001. 411(6841): p. 1058-64.
157. Buhtoiarov, I. N., et al., CD40 ligation activates murine macrophages via an IFN-gamma-dependent mechanism resulting in tumor cell destruction in vitro. J Immunol, 2005. 174(10): p. 6013-22.
158. Egilmez, N. K., et al., Human CD4+ effector T cells mediate indirect interleukin-12- and interferon-gamma-dependent suppression of autologous HLA-negative lung tumor xenografts in severe combined immunodeficient mice. Cancer Res, 2002. 62(9): p. 2611-7.
159. Pace, J. L., et al., Recombinant mouse gamma interferon induces the priming step in macrophage activation for tumor cell killing. J Immunol, 1983. 130(5): p. 2011-3.
160. Heusinkveld, M., et al., M2 macrophages induced by prostaglandin E2 and IL-6 from cervical carcinoma are switched to activated M1 macrophages by CD4+ Th1 cells. J Immunol, 2011. 187(3): p. 1157-65.
161. Hu, G. and S. Wang, Tumor-infiltrating CD45RO+ Memory T Lymphocytes Predict Favorable Clinical Outcome in Solid Tumors. Sci Rep, 2017. 7(1): p. 10376.
162. Lohneis, P., et al., Cytotoxic tumour-infiltrating T lymphocytes influence outcome in resected pancreatic ductal adenocarcinoma. Eur J Cancer, 2017. 83: p. 290-301.
163. Liu, S., et al., Role of Cytotoxic Tumor-Infiltrating Lymphocytes in Predicting Outcomes in Metastatic HER2-Positive Breast Cancer: A Secondary Analysis of a Randomized Clinical Trial. JAMA Oncol, 2017: p. e172085.
164. Berntsson, J., et al., The clinical impact of tumour-infiltrating lymphocytes in colorectal cancer differs by anatomical subsite: A cohort study. Int J Cancer, 2017. 141(8): p. 1654-1666.
165. Xu, Y., et al., Higher Numbers of T-Bet+ Tumor-Infiltrating Lymphocytes Associate with Better Survival in Human Epithelial Ovarian Cancer. Cell Physiol Biochem, 2017. 41(2): p. 475-483.
166. Melief, S. M., et al., Long-term Survival and Clinical Benefit from Adoptive T-cell Transfer in Stage IV Melanoma Patients Is Determined by a Four-Parameter Tumor Immune Signature. Cancer Immunol Res, 2017. 5(2): p. 170-179.
167. Scurr, M. J., et al., Low-dose cyclophosphamide induces anti-tumor T-cell responses which associate with survival in metastatic colorectal cancer. Clin Cancer Res, 2017.
168. Wang, L., et al., Arsenic trioxide is an immune adjuvant in liver cancer treatment. Mol Immunol, 2017. 81: p. 118-126.
169. Ouyang, Z., et al., Regulatory T cells in the immunotherapy of melanoma. Tumour Biol, 2016. 37(1): p. 77-85.
170. Dimeloe, S., et al., Human regulatory T cells lack the cyclophosphamide-extruding transporter ABCB1 and are more susceptible to cyclophosphamide-induced apoptosis. Eur J Immunol, 2014. 44(12): p. 3614-20.
171. Camisaschi, C., et al., Effects of cyclophosphamide and IL-2 on regulatory CD4+ T cell frequency and function in melanoma patients vaccinated with HLA-class I peptides: impact on the antigen-specific T cell response. Cancer Immunol Immunother, 2013. 62(5): p. 897-908.
172. Kan, S., et al., Suppressive effects of cyclophosphamide and gemcitabine on regulatory T-cell induction in vitro. Anticancer Res, 2012. 32(12): p. 5363-9.
173. Farsam, V., et al., Antitumor and immunomodulatory properties of artemether and its ability to reduce CD4+ CD25+ FoxP3+ T reg cells in vivo. Int Immunopharmacol, 2011. 11(11): p. 1802-8.
174. Stockmann, C., et al., The impact of the immune system on tumor: angiogenesis and vascular remodeling. Front Oncol, 2014. 4: p. 69.
175. Hirst, D. G., J. Denekamp, and B. Hobson, Proliferation kinetics of endothelial and tumour cells in three mouse mammary carcinomas. Cell Tissue Kinet, 1982. 15(3): p. 251-61.
176. Denekamp, J. and B. Hobson, Endothelial-cell proliferation in experimental tumours. Br J Cancer, 1982. 46(5): p. 711-20.
177. Hobson, B. and J. Denekamp, Endothelial proliferation in tumours and normal tissues: continuous labelling studies. Br J Cancer, 1984. 49(4): p. 405-13.
178. Folkman, J., Tumor angiogenesis: therapeutic implications. N Engl J Med, 1971. 285(21): p. 1182-6.
179. Folkman, J., Fighting cancer by attacking its blood supply. Sci Am, 1996. 275(3): p. 150-4.
180. Ellis, L. M. and I. J. Fidler, Finding the tumor copycat. Therapy fails, patients don't. Nat Med, 2010. 16(9): p. 974-5.
181. Wagner, S. C., et al., Cancer anti-angiogenesis vaccines: Is the tumor vasculature antigenically unique? J Transl Med, 2015. 13: p. 340.
182. Wei, Y. Q., et al., Immunotherapy of tumors with xenogeneic endothelial cells as a vaccine. Nat Med, 2000. 6(10): p. 1160-6.
183. Zhang, W., J. N. Liu, and X. Y. Tan, Vaccination with xenogeneic tumor endothelial proteins isolated in situ inhibits tumor angiogenesis and spontaneous metastasis. Int J Cancer, 2009. 125(1): p. 124-32.
184. Luo, Y., et al., Immunotherapy of tumors with protein vaccine based on chicken homologous Tie-2. Clin Cancer Res, 2006. 12(6): p. 1813-9.
185. He, Q. M., et al., Inhibition of tumor growth with a vaccine based on xenogeneic homologous fibroblast growth factor receptor-1 in mice. J Biol Chem, 2003. 278(24): p. 21831-6.
186. Wagner, S. C., et al., Safety of targeting tumor endothelial cell antigens. J Transl Med, 2016. 14: p. 90.
187. Ichim, T. E., et al., Induction of tumor inhibitory anti-angiogenic response through immunization with interferon Gamma primed placental endothelial cells: ValloVax. J Transl Med, 2015. 13: p. 90.
188. Catalona, W. J., et al., Identification of complement-receptor lymphocytes (B cells) in lymph nodes and tumor infiltrates. J Urol, 1975. 114(6): p. 915-21.
189. Wolf, G. T., et al., Lymphocyte subpopulations infiltrating squamous carcinomas of the head and neck: correlations with extent of tumor and prognosis. Otolaryngol Head Neck Surg, 1986. 95(2): p. 142-52.
190. Holmes, E. C., Immunology of tumor infiltrating lymphocytes. Ann Surg, 1985. 201(2): p. 158-63.
191. Lipponen, P. K., et al., Tumour infiltrating lymphocytes as an independent prognostic factor in transitional cell bladder cancer. Eur J Cancer, 1992. 29A(1): p. 69-75.
192. Di Giorgio, A., et al., The influence of tumor lymphocytic infiltration on long term survival of surgically treated colorectal cancer patients. Int Surg, 1992. 77(4): p. 256-60.
193. Kawata, A., et al., Tumor-infiltrating lymphocytes and prognosis of hepatocellular carcinoma. Jpn J Clin Oncol, 1992. 22(4): p. 256-63.
194. Furihata, M., et al., Prognostic significance of simultaneous infiltration of HLA-DR-positive dendritic cells and tumor infiltrating lymphocytes into human esophageal carcinoma. Tohoku J Exp Med, 1993. 169(3): p. 187-95.
195. Sarioglu, T., et al., The effect of lymphocytic infiltration on clinical survival in cancer of the tongue. Eur Arch Otorhinolaryngol, 1994. 251(6): p. 366-9.
196. Slootweg, P. J., et al., Lymphocytes at tumor margins in patients with head and neck cancer. Relationship with tumor size, human lymphocyte antigen molecules, and metastasis. Int J Oral Maxillofac Surg, 1994. 23(5): p. 286-9.
197. Mihm, M. C., Jr., C. G. Clemente, and N. Cascinelli, Tumor infiltrating lymphocytes in lymph node melanoma metastases: a histopathologic prognostic indicator and an expression of local immune response. Lab Invest, 1996. 74(1): p. 43-7.
198. Clemente, C. G., et al., Prognostic value of tumor infiltrating lymphocytes in the vertical growth phase of primary cutaneous melanoma. Cancer, 1996. 77(7): p. 1303-10.
199. Hakansson, A., et al., Tumour-infiltrating lymphocytes in metastatic malignant melanoma and response to interferon alpha treatment. Br J Cancer, 1996. 74(5): p. 670-6.
200. Eerola, A. K., Y. Soini, and P. Paakko, Tumour infiltrating lymphocytes in relation to tumour angiogenesis, apoptosis and prognosis in patients with large cell lung carcinoma. Lung Cancer, 1999. 26(2): p. 73-83.
201. Prince, M. E. P., et al., Evaluation of the immunogenicity of ALDH(high) human head and neck squamous cell carcinoma cancer stem cells in vitro. Oral Oncol, 2016. 59: p. 30-42.
202. Huang, Z., et al., Correlation of cancer stem cell markers and immune cell markers in resected non-small cell lung cancer. J Cancer, 2017. 8(16): p. 3190-3197.
203. Codd, A. S., et al., Cancer stem cells as targets for immunotherapy. Immunology, 2017.
204. Rapp, C., et al., Identification of T cell target antigens in glioblastoma stem-like cells using an integrated proteomics-based approach in patient specimens. Acta Neuropathol, 2017. 134(2): p. 297-316.
205. Maccalli, C., G. Parmiani, and S. Ferrone, Immunomodulating and Immunoresistance Properties of Cancer-Initiating Cells: Implications for the Clinical Success of Immunotherapy. Immunol Invest, 2017. 46(3): p. 221-238.
206. Meng, M. B., et al., Enhanced radioresponse with a novel recombinant human endostatin protein via tumor vasculature remodeling: experimental and clinical evidence. Radiother Oncol, 2013. 106(1): p. 130-7.
207. Czarnota, G. J., et al., Tumor radiation response enhancement by acoustical stimulation of the vasculature. Proc Natl Acad Sci USA, 2012. 109(30): p. E2033-41.
208. Peng, F., et al., Recombinant human endostatin normalizes tumor vasculature and enhances radiation response in xenografted human nasopharyngeal carcinoma models. PLoS One, 2012. 7(4): p. e34646.
209. Zawaski, J. A., et al., Effects of irradiation on brain vasculature using an in situ tumor model. Int J Radiat Oncol Biol Phys, 2012. 82(3): p. 1075-82.
210. Truman, J. P., et al., Endothelial membrane remodeling is obligate for anti-angiogenic radiosensitization during tumor radiosurgery. PLoS One, 2010. 5(9).
211. Brose, K., et al., Slit proteins bind Robo receptors and have an evolutionarily conserved role in repulsive axon guidance. Cell, 1999. 96(6): p. 795-806.
212. Yadav, S. S. and G. Narayan, Role of ROBO4 signalling in developmental and pathological angiogenesis. Biomed Res Int, 2014. 2014: p. 683025.
213. Zhuang, X., et al., Robo4 vaccines induce antibodies that retard tumor growth. Angiogenesis, 2014.
214. Paprocka, M., et al., CD133 positive progenitor endothelial cell lines from human cord blood. Cytometry A, 2011. 79(8): p. 594-602.
215. Chen, C., et al., Adult endothelial progenitor cells retain hematopoiesis potential. Transplant Proc, 2010. 42(9): p. 3745-9.
216. Smadja, D. M., et al., Increased VEGFR2 expression during human late endothelial progenitor cells expansion enhances in vitro angiogenesis with up-regulation of integrin alpha(6). J Cell Mol Med, 2007. 11(5): p. 1149-61.
217. Ziegler, B. L., et al., KDR receptor: a key marker defining hematopoietic stem cells. Science, 1999. 285(5433): p. 1553-8.
218. Yan, H. X., et al., Active immunotherapy for mouse breast cancer with irradiated whole-cell vaccine expressing VEGFR2. Oncol Rep, 2013. 29(4): p. 1510-6.
219. Feng, K., et al., Anti-angiogenesis effect on glioma of attenuated Salmonella typhimurium vaccine strain with flk-1 gene. J Huazhong Univ Sci Technolog Med Sci, 2004. 24(4): p. 389-91.
220. Wada, S., et al., Rationale for antiangiogenic cancer therapy with vaccination using epitope peptides derived from human vascular endothelial growth factor receptor 2. Cancer Res, 2005. 65(11): p. 4939-46.
221. Yan, J., et al., A promising new approach of VEGFR2-based DNA vaccine for tumor immunotherapy. Immunol Lett, 2009. 126(1-2): p. 60-6.
222. Ren, S., et al., Inhibition of tumor angiogenesis in lung cancer by T4 phage surface displaying mVEGFR2 vaccine. Vaccine, 2011. 29(34): p. 5802-11.
223. Wei, Y., et al., Enhancement of DNA vaccine efficacy by targeting the xenogeneic human chorionic gonadotropin, survivin and vascular endothelial growth factor receptor 2 combined tumor antigen to the major histocompatibility complex class II pathway. J Gene Med, 2012. 14(5): p. 353-62.
224. Suzuki, H., et al., Multiple therapeutic peptide vaccines consisting of combined novel cancer testis antigens and anti-angiogenic peptides for patients with non-small cell lung cancer. J Transl Med, 2013. 11: p. 97.
225. Facciponte, J. G., et al., Tumor endothelial marker 1-specific DNA vaccination targets tumor vasculature. J Clin Invest, 2014. 124(4): p. 1497-511.
226. Lladser, A., et al., Intradermal DNA electroporation induces survivin-specific CTLs, suppresses angiogenesis and confers protection against mouse melanoma. Cancer Immunol Immunother, 2010. 59(1): p. 81-92.
227. Xiang, R., et al., Oral DNA vaccines target the tumor vasculature and microenvironment and suppress tumor growth and metastasis. Immunol Rev, 2008. 222: p. 117-28.
228. Xiang, R., et al., A DNA vaccine targeting survivin combines apoptosis with suppression of angiogenesis in lung tumor eradication. Cancer Res, 2005. 65(2): p. 553-61.
229. Valdez, Y., M. Maia, and E. M. Conway, CD248: reviewing its role in health and disease. Curr Drug Targets, 2012. 13(3): p. 432-9.
230. Plum, S. M., et al., Generation of a specific immunological response to FGF-2 does not affect wound healing or reproduction. Immunopharmacol Immunotoxicol, 2004. 26(1): p. 29-41.
231. Wei, Y. Q., et al., Immunogene therapy of tumors with vaccine based on Xenopus homologous vascular endothelial growth factor as a model antigen. Proc Natl Acad Sci USA, 2001. 98(20): p. 11545-50.
232. Liu, J. Y., et al., Immunotherapy of tumors with vaccine based on quail homologous vascular endothelial growth factor receptor-2. Blood, 2003. 102(5): p. 1815-23.
233. Su, J. M., et al., Active immunogene therapy of cancer with vaccine on the basis of chicken homologous matrix metalloproteinase-2. Cancer Res, 2003. 63(3): p. 600-7.
234. Tan, G. H., et al., Active immunotherapy of tumors with a recombinant xenogeneic endoglin as a model antigen. Eur J Immunol, 2004. 34(7): p. 2012-21.
235. Jiao, J. G., et al., A plasmid DNA vaccine encoding the extracellular domain of porcine endoglin induces anti-tumour immune response against self-endoglin-related angiogenesis in two liver cancer models. Dig Liver Dis, 2006. 38(8): p. 578-87.

1. A method of inducing a T cell and/or B cell response to tumor associated endothelial cells and/or tumor vascular channels comprising the steps of: a) selecting a pluripotent stem cell; b)modifying said pluripotent stem cell so as to induce expression of an immunogenic molecule; c) modifying said pluripotent stem cell so as to induce loss of expression of an immunosuppressive molecule; d) modifying said pluripotent stem cell so as to induce loss of expression of an immune suppressive signaling molecule; e) inducing differentiation of said pluripotent stem cell into endothelial cells under conditions which replicate the tumor microenvironment; f) obtaining said endothelial cells differentiated under conditions that replicate said tumor microenvironment and substantially isolating said cells in order to obtain a relatively homogeneous population of cells which resemble tumor endothelium associated cells; g) optionally mitotically inactivating said cells; and h) administering said cells in a manner to stimulate an immune response.

2. The method of claim 1, wherein said endothelial-like cell as transfected with GM-CSF.

3. The method of claim 2, wherein said transfection with GM-CSF is sufficient to induce differentiation of dendritic cells in proximity to said administered endothelial-like cell.

4. The method of claim 3, wherein said dendritic cells express CD40.

5. The method of claim 3, wherein said dendritic cells express CD11c.

6. The method of claim 3, wherein said dendritic cells express CD83.

7. The method of claim 3, wherein said dendritic cells express CD80.

8. The method of claim 3, wherein said dendritic cells express CD86.

9. The method of claim 1, wherein said endothelial-like cells are co-administered with monocytes.

10. The method of claim 9, wherein said monocytes are allogeneic to the recipient.

11. The method of claim 9, wherein said monocytes are autologous to the recipient.

12. The method of claim 9, wherein said monocytes are xenogeneic to the recipient.

13. The method of claim 9, wherein said monocytes are transfected with interleukin-1.

14. The method of claim 9, wherein said monocytes are induced to produce interleukin-1.

15. The method of claim 14, wherein said monocytes are induced to produce interleukin-1 by incubation with a toll like receptor agonist.

16. The method of claim 15, wherein said toll like receptor is toll like receptor 4.

17. The method of claim 16, wherein said toll like receptor 4 is activated by lipopolysaccharide.

18. The method of claim 16, wherein said toll like receptor 4 is activated by HMGB-1.

19. The method of claim 16, wherein said toll like receptor 4 is activated by beta glucan.

20. The method of claim 16, wherein said toll like receptor 4 is activated by BCG.

21. The method of claim 16, wherein said toll like receptor 4 is activated by neutrophil extracellular traps.
